SEQUENCE LISTING (12 sequences)

SEQ ID NO: 1 - DNA, 1824 bp, canine distemper virus
  misc_feature: H gene
atgctctcctaccaagacaaggtgggtgccttctataaagataatgcaagagctaattcatccaagctgtccttagtgacagaagagcaagggggcaggagaccaccctatttgctgtttgtccttctcatcctactggttggaatcatggccttacttgctatcactggagttcgatttcaccaagtatcaactagcaatatggaatttagcagattgctgaaagaggatatggagaaatcagaggccgtacatcaccaagtcatagatgtcttgacaccgctcttcaaaattattggagatgagattgggttacggttgccacaaaaactaaacgagatcaaacaatttatccttcaaaagacaaacttcttcaatccgaacagggaattcgacttccgcgatctccactggtgcattaacccacctagtaagatcaaggtgaatttaactaattactgcgatacaattgggctcagaaaatctattgcatcggcagcaaatcccatccttttatcagcactctccagaggcagaggtgacatattcccaccatacagatgcagtggagctactacttcagtaggcagatttttccccctatcagtatcattgtccatgtctttgatctcaagaacatcagagataatcaatatgctaaccgctatctcagacggagtgtatggtaaaacttatttgctagtgcctgattatattgaaggggagttcgacacgcaaaagattcgagtctttgagatagggttcatcaaacggtggctgaatgacatgccattactccagacaaccaactatatggtcctcccggagaattccaaagccaaggtatgtactatagcagtgggcgagttgacactggcttccttgtgtgtagatgagagcaccgtattgttatatcatgacagcaatggttcacaagatggtattctagtagtgacgctgggaatatttggggcaacacctatggatcaagttgaagaggtgatacctgtcgctcacccatcagtagaaaaaatacatataacaaatcaccgtgggttcataaaagattcaatagcaacctggatggtgcctgcattggtctctgagaaacaagaggaacaaaaaaattgtctggagtcggcttgtcaaagaaaatcctaccctatgtgcaaccaaacgtcatgggaaccctttggaggaggacagttgccatcttatgggcggttgacattacctctagatccaagtattgaccttcaacttaacatatcgtttacatacggtccggttatactgaatggagacggtatggattattatgaaagcccacttttggactccggatggcttaccattcctcccaagaacggaacagtccttggattgataaacaaagcaagtagaggagaccagttcactgtaatcccccatgtgttgacatttgcgcccagggaatcaagtggaaattgttatttacctattcaaacattccagattatggataaagatgtccttactgagtccaatttagtggtgttgcctacacagaattttagatatgtcatagcaacatatgatatatcccgggacgatcatgcgattgtttattatgtttatgacccaatccggaagatttcttttacgtacccatttagactaactaccaagggtagacctgatttcctaaggattgaatgttttgtgtgggatgacgatttgtggtgtcaccaattttaccgattcgaggctgacatcaccaactttacaaccagtgttgagaatttagtccgtataagattctcatgtaaccgttcaaaaccttga

SEQ ID NO: 2 - DNA, 1989 bp, canine distemper virus
  misc_feature: F gene
atgcacaacaaaatccccaaaagatccaaaacccaaacacacacccaacaagacctccccccacaacacagcaccaaatccgccgagaccaagacctcccaagcacgacacagcataacatcggctcagcgatccacgcaccatggtcctcgaacatcagacaggcccgtccactacatcatgaacaggaccaggtcttgcaagcaagccagctacagatcggataacatcccggctcacggagaccacgagggtattatccatcacacaccagagagtgtctcccaaggagcgagatcccggttcaaaaggcggcaatccaatgcaaccaactcaggctctcagtgcacctggttagtcctgtggtgcatcggaatagccagtctctttctttgttctaaggctcagatacattggaataatttgtcaactattgggattatcgggactgacagtgtccattataagatcatgactaggcccagtcaccagtacttggtcataaaactaatgcctaatgtttcacttatagataattgtaccaaagcagaattaggtgagtatgagaaattattaaattcagtactcgagccaatcaaccaagctttgactctaatgaccaagaatgtgaagcccctacagtcagtagggtcaggtaggagacaaaggcgttttgcaggagtggtgcttgcaggtgcagccttaggagtggccacagctgcacaaatcactgcagggatagctttacatcaatccaacctcaatgctcaagcaatccaatctctgagaactagccttgaacagtccaacaaggctatagaagaaattagggaggcaacccaggaaaccgtcattgccgttcagggagtccaggattacgtcaataatgaacttgtccctgctatgcaacatatgtcgtgtgaattagttgggcagagattagggttaaaactgcttaggtattataccgagttgttgtcaatatttggcccgagtttacgtgaccctatttcagccgagatatcaattcaagcactgagttatgcccttgggggagaaattcataagatacttgagaagttgggatattctggaaatgatatgattgcaattttggagagtcgggggataaaaacaaaaataactcatgttgatctccccgggaaactcatcatcttaagtatctcatacccaactttatcagaagtcaagggggttatagtccacagactggaagcagtttcttataacatagggtcacaggagtggtacaccactgtcccgaggtatgttgcaactaatggttacttaatatctaattttgatgagtcatcctgtgtattcgtctcagaatcagccatttgtagccagaactccctataccccatgagcccgcttctacaacaatgcattaggggcgacacttcatcttgtgctcggaccttggtgtctgggactatgggcaacaagtttattctgtcaaaaggtaatatcgtcgcaaattgtgcttctatactatgtaagtgttatagcacaagcacaattatcaatcagagtcctgataagttgctgacatttattgcctccgatacctgcccactggttgaaatagatggtgtaactatccaggttggagggaggcaataccctgatatggtatacgaaagcaaagttgccttaggccctgctatatcacttgagaggttagatgtaggtacaaatttaggaaacgcccttaagaaactggatgatgctaaggtactgatagactcctctaaccagatccttgagacggttaggcgctcttcctttcattttggcagtctcctcagcgttcccatattaatctgtacagccctggctttgttgttgctgatttactgctgtaaaagacgctaccaacggacattcaagcagaatactaaggtcgatccgacatttaaacctgatttgactggaacttcgaaatcctatgtaagatcactctga

SEQ ID NO: 3 - DNA, 7975 bp, Artificial Sequence
  plasmid pdV-CDV-H
tatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggcccgtggccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcccagctggcttatcgaaattaatacgactcactatagggagaccggaatttgcaggatcgcgctgctgtacctgaagatatacgagctcccgggctgcacgaacttcctgcacgtggacctgaagcccgacaacgtgctcatcttcgacagcgcgcgcgcgctcagcgtgactgcggccggtgcgacttttcgcttcgaagagcccgtgcgcgcggcgctgaacgacttcgacttcgcgcgcgtggccaccatcgagaaccgcaagatcgcgggcagcgtccgcgtgccgcagaactggtactacgacttccacttcttcgcgcacacgctgctgcgcgcgtacccgcacatcgccgcggaggacccgggcttccacgcgctgctctcggagctcacggtctcgtgctcgcgcgggacctgcgaccgcttccggctgcgcgtgtcctcgccgcaccccatcgagcacctcgcgcggctggtgcgccgcgacgtcttctcccgctggataaatgccgccgcggacgcccccgacgccgcactctcctgagcccacgcccgcggcgccgggctcgctgtacgacgtcttcctcgcgcgcttcctgcgccagctggccgcgcgcgcggcgccggcctcggccgcctgcgccgtgcgcgtgggtgcggtgcgcggccgcctgcggaactgcgagctggtggtgctgaaccgctgccacgcggacgctgccggcgcgctcgcgctggcctccgcggcgctggcggaaacgctggcggagctgccgcgcgcggacaggctcgccgtcgcgcgcgagctgggcgtggacccagagcacccggagctgacgccggaccccgcctgcgcgggcgagagcgcgcttgcgcagaacatcgacatccagacgctggacctgggcgactgcggcgaccccaaaggccgccgactgcgcgtggcgctggtgaacagcggccacgcggccgcaaactgcgcgctcgcgcgcgtagcgaccgcgctgacgcgccgcgtgcccgcaagccggcacggcctcgcggagggcggcacgccgccgtggacgctgctgctggcggtggccgcggtgacggtgctcagcgtggtggcggtttcgctgctgcggcgcgcgctgcgggtgcgctaccaattcgcgcggccggccgcgctgcgcgcgtagccgcgcaaaatgtaaattataacgcccatcccgggataagcttgatatctgcaggatcccaggtagtccaacaatgctctcctaccaagacaaggtgggtgccttctataaagataatgcaagagctaattcatccaagctgtccttagtgacagaagagcaagggggcaggagaccaccctatttgctgtttgtccttctcatcctactggttggaatcatggccttacttgctatcactggagttcgatttcaccaagtatcaactagcaatatggaatttagcagattgctgaaagaggatatggagaaatcagaggccgtacatcaccaagtcatagatgtcttgacaccgctcttcaaaattattggagatgagattgggttacggttgccacaaaaactaaacgagatcaaacaatttatccttcaaaagacaaacttcttcaatccgaacagggaattcgacttccgcgatctccactggtgcattaacccacctagtaagatcaaggtgaatttaactaattactgcgatacaattgggctcagaaaatctattgcatcggcagcaaatcccatccttttatcagcactctccagaggcagaggtgacatattcccaccatacagatgcagtggagctactacttcagtaggcagatttttccccctatcagtatcattgtccatgtctttgatctcaagaacatcagagataatcaatatgctaaccgctatctcagacggagtgtatggtaaaacttatttgctagtgcctgattatattgaaggggagttcgacacgcaaaagattcgagtctttgagatagggttcatcaaacggtggctgaatgacatgccattactccagacaaccaactatatggtcctcccggagaattccaaagccaaggtatgtactatagcagtgggcgagttgacactggcttccttgtgtgtagatgagagcaccgtattgttatatcatgacagcaatggttcacaagatggtattctagtagtgacgctgggaatatttggggcaacacctatggatcaagttgaagaggtgatacctgtcgctcacccatcagtagaaaaaatacatataacaaatcaccgtgggttcataaaagattcaatagcaacctggatggtgcctgcattggtctctgagaaacaagaggaacaaaaaaattgtctggagtcggcttgtcaaagaaaatcctaccctatgtgcaaccaaacgtcatgggaaccctttggaggaggacagttgccatcttatgggcggttgacattacctctagatccaagtattgaccttcaacttaacatatcgtttacatacggtccggttatactgaatggagacggtatggattattatgaaagcccacttttggactccggatggcttaccattcctcccaagaacggaacagtccttggattgataaacaaagcaagtagaggagaccagttcactgtaatcccccatgtgttgacatttgcgcccagggaatcaagtggaaattgttatttacctattcaaacattccagattatggataaagatgtccttactgagtccaatttagtggtgttgcctacacagaattttagatatgtcatagcaacatatgatatatcccgggacgatcatgcgattgtttattatgtttatgacccaatccggaagatttcttttacgtacccatttagactaactaccaagggtagacctgatttcctaaggattgaatgttttgtgtgggatgacgatttgtggtgtcaccaattttaccgattcgaggctgacatcaccaactttacaaccagtgttgagaatttagtccgtataagattctcatgtaaccgttcaaaaccttgacagtatgatgatacaggtaccgaattcatcgtcgacttcgagagcttagaatgcgtccccacggaagaggcaaacgtaacgatgcaactcatgggagcgtcggtctccggtggtaacgggatgcaacatctgagcttcgtagagcataagaaatgcgattgtaaaccaccactcacgaccacgccaccgacgaccacaaggccgcccagaagacgccgctagaactttttatggaccgcatatccaaacgatgatgcgatcaggtcatgcggaaggaggctccacggagcaaagtgaaaaaggaccgcctagagtcgagacccctccctcccgcctcgggcaaacccacagccgccgcaaacaccacacccgccgacctaccatgcacccctcgccgcgccggctgctcggcgcgctcgcgctgctggcgctgggcttcgctcggcgcgctcttcgccccgcggcgccgctcgtgccggccgccttcctggaggtggggcacgtgcgcgcgaacccgtccgcctcggtgacctgcctcacggtgggcggcgacgggcggcacatggcggcggtcgcgcacggcggcgggacgctctcgccggtgtacccgctggccgccggcatgcacgcgaccttctcctccgcgcgcaagggcgcgctgctgctgaacgtcgcgaccgtgactgtgtacgacgtgcgcgcgctcgcccccgagttcgagctcgtctgcatcgcggtggtcggcggctacaactcggccgcggccgccacgcggcccgcggccgagtggcaccgccagctggagctgcgccgctcggagctgtgacccctccctccccggtctccctctgtctttgtaatcggccttagagattagacatcatcctccacgcctctttgtccgccgcccttcttcgcggacggatgaaccaattaattaattatttttgtcgctcgcccgctcactccggcaagggaacgagtgacgttaactctctcaccctcacgcacaagaacaagaaccgctcactcaccgggcaagggaacacggttaaggtcaactcactcgcgagaacaagttgaccctcactctagagaacgaggaacgggcaacaagcaaccgtcaactcacttaccacgagaacaagttgaccgccactcaaagggaacagagaacagtaaccgttctcgctcgctcggaacaatagaacaagttaacgtcaactcgctcgctcggtgtaagagaacaacagaacaagcaactgttgaccactcaacccccggagaagagaacaagagagcagtcaactcacccactcagtcttggatgagaggaggacgagttaacgagtactcgcacgcagagtgagagagtgaggacataataatagttaacgagttaatactcactcgctcactcagagtgagagagaaccagtgagcgagttaaccgcgcacacgagcgagagaacagtgaactgctcgcgcgctcgctcggtagcagtcggcctttcttaaaacggttcgtaaaacttttcccgagacagttcaccctccaaaacttttaaaactaaactcggaggtggcctgccctccactctccgtaaaacttttgtaaaactgtcggaggtcggtcgacttcgcaactcgtccgcgaaaacttttcgtgggcagtgtctgcctctctcaggctcctcgcatcactttcgcggagcctcgaggtaggtcacctctctccaaacttttgtaaaaactttttcgcggagcctctggaggccgtcctccctccaaaacttttcgtaaaatctcttcggaggccgtcctccctccaaaacttttcgtaaaatctttgggaggtcgacctccctcaaaactttttataaagtagcttgtattc

SEQ ID NO: 4 - DNA, 8134 bp, Artificial Sequence
  plasmid pdV-CDV-F
tatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggcccgtggccgggggactgttgggcgccatctccttgcatgcaccattccttgcggcggcggtgctcaacggcctcaacctactactgggctgcttcctaatgcaggagtcgcataagggagagcgtcgatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcccagctggcttatcgaaattaatacgactcactatagggagaccggaatttgcaggatcgcgctgctgtacctgaagatatacgagctcccgggctgcacgaacttcctgcacgtggacctgaagcccgacaacgtgctcatcttcgacagcgcgcgcgcgctcagcgtgactgcggccggtgcgacttttcgcttcgaagagcccgtgcgcgcggcgctgaacgacttcgacttcgcgcgcgtggccaccatcgagaaccgcaagatcgcgggcagcgtccgcgtgccgcagaactggtactacgacttccacttcttcgcgcacacgctgctgcgcgcgtacccgcacatcgccgcggaggacccgggcttccacgcgctgctctcggagctcacggtctcgtgctcgcgcgggacctgcgaccgcttccggctgcgcgtgtcctcgccgcaccccatcgagcacctcgcgcggctggtgcgccgcgacgtcttctcccgctggataaatgccgccgcggacgcccccgacgccgcactctcctgagcccacgcccgcggcgccgggctcgctgtacgacgtcttcctcgcgcgcttcctgcgccagctggccgcgcgcgcggcgccggcctcggccgcctgcgccgtgcgcgtgggtgcggtgcgcggccgcctgcggaactgcgagctggtggtgctgaaccgctgccacgcggacgctgccggcgcgctcgcgctggcctccgcggcgctggcggaaacgctggcggagctgccgcgcgcggacaggctcgccgtcgcgcgcgagctgggcgtggacccagagcacccggagctgacgccggaccccgcctgcgcgggcgagagcgcgcttgcgcagaacatcgacatccagacgctggacctgggcgactgcggcgaccccaaaggccgccgactgcgcgtggcgctggtgaacagcggccacgcggccgcaaactgcgcgctcgcgcgcgtagcgaccgcgctgacgcgccgcgtgcccgcaagccggcacggcctcgcggagggcggcacgccgccgtggacgctgctgctggcggtggccgcggtgacggtgctcagcgtggtggcggtttcgctgctgcggcgcgcgctgcgggtgcgctaccaattcgcgcggccggccgcgctgcgcgcgtagccgcgcaaaatgtaaattataacgcccatcccgggataagcttgatatctgcaggatccacagccgagccccatgcacaacaaaatccccaaaagatccaaaacccaaacacacacccaacaagacctccccccacaacacagcaccaaatccgccgagaccaagacctcccaagcacgacacagcataacatcggctcagcgatccacgcaccatggtcctcgaacatcagacaggcccgtccactacatcatgaacaggaccaggtcttgcaagcaagccagctacagatcggataacatcccggctcacggagaccacgagggtattatccatcacacaccagagagtgtctcccaaggagcgagatcccggttcaaaaggcggcaatccaatgcaaccaactcaggctctcagtgcacctggttagtcctgtggtgcatcggaatagccagtctctttctttgttctaaggctcagatacattggaataatttgtcaactattgggattatcgggactgacagtgtccattataagatcatgactaggcccagtcaccagtacttggtcataaaactaatgcctaatgtttcacttatagataattgtaccaaagcagaattaggtgagtatgagaaattattaaattcagtactcgagccaatcaaccaagctttgactctaatgaccaagaatgtgaagcccctacagtcagtagggtcaggtaggagacaaaggcgttttgcaggagtggtgcttgcaggtgcagccttaggagtggccacagctgcacaaatcactgcagggatagctttacatcaatccaacctcaatgctcaagcaatccaatctctgagaactagccttgaacagtccaacaaggctatagaagaaattagggaggcaacccaggaaaccgtcattgccgttcagggagtccaggattacgtcaataatgaacttgtccctgctatgcaacatatgtcgtgtgaattagttgggcagagattagggttaaaactgcttaggtattataccgagttgttgtcaatatttggcccgagtttacgtgaccctatttcagccgagatatcaattcaagcactgagttatgcccttgggggagaaattcataagatacttgagaagttgggatattctggaaatgatatgattgcaattttggagagtcgggggataaaaacaaaaataactcatgttgatctccccgggaaactcatcatcttaagtatctcatacccaactttatcagaagtcaagggggttatagtccacagactggaagcagtttcttataacatagggtcacaggagtggtacaccactgtcccgaggtatgttgcaactaatggttacttaatatctaattttgatgagtcatcctgtgtattcgtctcagaatcagccatttgtagccagaactccctataccccatgagcccgcttctacaacaatgcattaggggcgacacttcatcttgtgctcggaccttggtgtctgggactatgggcaacaagtttattctgtcaaaaggtaatatcgtcgcaaattgtgcttctatactatgtaagtgttatagcacaagcacaattatcaatcagagtcctgataagttgctgacatttattgcctccgatacctgcccactggttgaaatagatggtgtaactatccaggttggagggaggcaataccctgatatggtatacgaaagcaaagttgccttaggccctgctatatcacttgagaggttagatgtaggtacaaatttaggaaacgcccttaagaaactggatgatgctaaggtactgatagactcctctaaccagatccttgagacggttaggcgctcttcctttcattttggcagtctcctcagcgttcccatattaatctgtacagccctggctttgttgttgctgatttactgctgtaaaagacgctaccaacggacattcaagcagaatactaaggtcgatccgacatttaaacctgatttgactggaacttcgaaatcctatgtaagatcactctgaagcactctggggtaccgaattcatcgtcgacttcgagagcttagaatgcgtccccacggaagaggcaaacgtaacgatgcaactcatgggagcgtcggtctccggtggtaacgggatgcaacatctgagcttcgtagagcataagaaatgcgattgtaaaccaccactcacgaccacgccaccgacgaccacaaggccgcccagaagacgccgctagaactttttatggaccgcatatccaaacgatgatgcgatcaggtcatgcggaaggaggctccacggagcaaagtgaaaaaggaccgcctagagtcgagacccctccctcccgcctcgggcaaacccacagccgccgcaaacaccacacccgccgacctaccatgcacccctcgccgcgccggctgctcggcgcgctcgcgctgctggcgctgggcttcgctcggcgcgctcttcgccccgcggcgccgctcgtgccggccgccttcctggaggtggggcacgtgcgcgcgaacccgtccgcctcggtgacctgcctcacggtgggcggcgacgggcggcacatggcggcggtcgcgcacggcggcgggacgctctcgccggtgtacccgctggccgccggcatgcacgcgaccttctcctccgcgcgcaagggcgcgctgctgctgaacgtcgcgaccgtgactgtgtacgacgtgcgcgcgctcgcccccgagttcgagctcgtctgcatcgcggtggtcggcggctacaactcggccgcggccgccacgcggcccgcggccgagtggcaccgccagctggagctgcgccgctcggagctgtgacccctccctccccggtctccctctgtctttgtaatcggccttagagattagacatcatcctccacgcctctttgtccgccgcccttcttcgcggacggatgaaccaattaattaattatttttgtcgctcgcccgctcactccggcaagggaacgagtgacgttaactctctcaccctcacgcacaagaacaagaaccgctcactcaccgggcaagggaacacggttaaggtcaactcactcgcgagaacaagttgaccctcactctagagaacgaggaacgggcaacaagcaaccgtcaactcacttaccacgagaacaagttgaccgccactcaaagggaacagagaacagtaaccgttctcgctcgctcggaacaatagaacaagttaacgtcaactcgctcgctcggtgtaagagaacaacagaacaagcaactgttgaccactcaacccccggagaagagaacaagagagcagtcaactcacccactcagtcttggatgagaggaggacgagttaacgagtactcgcacgcagagtgagagagtgaggacataataatagttaacgagttaatactcactcgctcactcagagtgagagagaaccagtgagcgagttaaccgcgcacacgagcgagagaacagtgaactgctcgcgcgctcgctcggtagcagtcggcctttcttaaaacggttcgtaaaacttttcccgagacagttcaccctccaaaacttttaaaactaaactcggaggtggcctgccctccactctccgtaaaacttttgtaaaactgtcggaggtcggtcgacttcgcaactcgtccgcgaaaacttttcgtgggcagtgtctgcctctctcaggctcctcgcatcactttcgcggagcctcgaggtaggtcacctctctccaaacttttgtaaaaactttttcgcggagcctctggaggccgtcctccctccaaaacttttcgtaaaatctcttcggaggccgtcctccctccaaaacttttcgtaaaatctttgggaggtcgacctccctcaaaactttttataaagtagcttgtattc

SEQ ID NO: 5 - DNA, 30 bp, Artificial Sequence
  primer ORF32N
gcgcgctgcgggtgcgctaccaattcgcgc

SEQ ID NO: 6 - DNA, 32 bp, Artificial Sequence
  primer ORF31N
gcatcccgttaccaccggagaccgacgctccc

SEQ ID NO: 7 - DNA, 24 bp, Artificial Sequence
  primer CDH-F
cagatgcagtggagctactacttc

SEQ ID NO: 8 - DNA, 24 bp, Artificial Sequence
  primer CDH-R
ggatctagaggtaatgtcaaccgc

SEQ ID NO: 9 - DNA, 26 bp, Artificial Sequence
  primer CDF-F
ccctgctatgcaacatatgtcgtgtg

SEQ ID NO: 10 - DNA, 28 bp, Artificial Sequence
  primer CDF-R
cgttataccatatcagggtattgcctcc

SEQ ID NO: 11 - DNA, 23 bp, Artificial Sequence
  primer lacZ-F
cgatactgtcgtcgtcccctcaa

SEQ ID NO: 12 - DNA, 22 bp, Artificial Sequence
  primer lacZ-R
caactcgccgcacatctgaact

(19) United States
(12) Patent Application Publication
(10) Pub. No.: US 2012/0020995 A1
(43) Pub. Date: Jan. 26, 2012

(54) PARAPOXVIRUS VECTORS

(75) Inventors: Olivier Michel Martinon, Richland, MI (US); Nanda Kumar D. Reddy, Holly, MI (US)

(73) Assignee: PFIZER INC., New York, NY (US)

(21) Appl. No.: 13/185,561

(22) Filed: Jul. 19, 2011

Related U.S. Application Data
(60) Provisional application No. 61/365,870, filed on Jul. 20, 2010.

(51) Int. Cl. A61K 39/275; A61P 31/14; A61P 37/04; C12N 7/01; C12Q 1/70

(52) U.S. Cl. 424/199.1; 435/235.1; 435/440; 435/5

(57) ABSTRACT

The present invention relates to recombinant parapoxviruses comprising heterologous DNA derived from a canine distemper virus, to the preparation of such constructs, and to their use in immunogenic compositions and vaccines. It further relates to the use of recombinant parapoxviruses for diagnostics.

### CROSS-REFERENCE TO RELATED APPLICATION

This application claims priority, under 35 U.S.C. §119(e), to U.S. Provisional Application Ser. No. 61/365,870, filed on Jul. 20, 2010, which is incorporated herein by reference.

### FIELD OF THE INVENTION

The present invention relates to recombinant parapoxviruses that contain heterologous DNA derived from a canine distemper virus (CDV) and to their use in immunogenic compositions and vaccines. It also relates to methods for vaccinating against, treating, or preventing disease caused by CDV. It further relates to the use of recombinant parapoxviruses for diagnostics.

### BACKGROUND OF THE INVENTION

Viruses of the Poxviridae family are oval, quite large, double-stranded DNA viruses. The genus Parapoxvirus (PPV) is included among these viruses. They measure about 220-300 nm long by 140-170 nm wide. They possess a unique spiral coat that distinguishes them from the other poxviruses.

The PPV are divided into three different species. However, it has still not been clarified whether these viruses are autonomous species within the parapoxvirus genus or whether they are the same species. The first species, Parapoxvirus ovis, (ORF virus, ORFV), is regarded as the prototype of the genus. It is also called ecthyma contagiosum virus, contagious pustular dermatitis virus or orf virus. The second, Parapoxvirus bovis 1, is also called bovine papular stomatitis virus or stomatitis papulosa virus. The third, Parapoxvirus bovis 2, is also called udderpoxvirus, paravaccinia virus, pseudocowpox virus or milker's nodule virus.

Parapoxvirus species are endemic in ruminants. PPVs have been found in red deer, reindeer, red squirrels, and harbor seals. Infections with PPV can cause local diseases in both animals and man. The zoonotic hosts of PPV species are sheep, goats, and cattle. They cause infections in humans through direct contact with infected animals, reacting with localized epidermal lesions which heal without scaring. Prophylactic measures, such as vaccines, can be used to control the diseases.

Vectors for expressing foreign genetic information based on avipox, racoonpox, capripox, swinepox, or vaccinia virus have been described previously (see U.S. Pat. No. 5,942,235 and U.S. Pat. No. 7,094,412). Parapoxviruses represent different candidates that can poxviruses cannot be used for Parapoxvirus. An example of such differences is that ORFV is missing a thymidine kinase (TK) gene, which is used for selection of recombinants in the different orthopoxviruses. Also, some poxviruses have the ability to agglutinate erythrocytes, which is mediated by way of a surface protein, the haemagglutinin, while Parapoxviruses do not.

PPV can have an immunomodulatory effect because they stimulate generalized (non-specific) immune reactions in vertebrates. They have been used successfully in veterinary medicine for increasing general resistance in animals. They can be combined with a homologous and/or heterologous antigen to provide vaccines that have a pathogen-specific effect which lasts for months to years, as well as a rapid non-pathogen-specific effect.

Parapoxvirus ovis has been used previously as a vector, as described in U.S. Pat. No. 6,365,393; Rziha et al., 2000, J. Biotechnol., 83, 137-145; WO 2004/054614; and Fischer et al., 2003, J. Virol. 77, 9312-9323. It offers remarkable advantages when used as a vector, including a very narrow host range, lack of systemic infection, short-term vector-specific immunity (allowing repeated immunizations), early vaccination (induction of immunity can be started in presence of maternal antibodies), and beneficial immune modulating properties. The present invention relates to using Parapoxvirus as a vector for heterologous DNA derived from canine distemper virus. Parapoxvirus ovis strain D1701 is a highly attenuated strain that can be propagated in cell culture with titers comparable to those of the wild type virus. It has outstanding immune stimulating properties both in hosts that support replication of the infectious vector virus (e.g., sheep and goats) and in hosts that do not (e.g., dogs, swine, horse, mouse, and rat). Zylexis®, formerly known as Baypamune®, which is a preparation of chemically inactivated Parapoxvirus ovis, derived from strain D1701, is used for the prophylaxis, metaphylaxis and therapeutic treatment of infectious diseases and for preventing stress-induced diseases in animals.

Canine distemper is a highly infectious, acute or subacute, febrile viral disease of dogs and other carnivores, which occurs world-wide. Some dogs show primarily respiratory signs, others intestinal signs, and at least 30% of the animals develop neurological symptoms. All experimentally infected dogs have histopathological lesions in the central nervous system. The mortality rate ranges between 30% and 80%. In a minority of cases, dogs that have recovered continue to harbor the virus in brain cells, where it replicates slowly and eventually produces old dog encephalitic. Dogs surviving distemper have life-long immunity to reinfection. Immunization is recommended for the control of distemper in dogs; annual re-vaccination is recommended.

Canine distemper is caused by canine distemper virus (CDV), a member of the genus Morbillivirus, and the family Paramyxoviridae. CDV is closely related to the viruses which cause measles and rinderpest. Canine distemper virions are enveloped, and contain a negative-strand RNA genome of 15,616 nucleotides. The entire genome has been sequenced for the cell culture-adapted Onderstepoort (OP-CDV) strain (Sidhu et al., 1993, Virology 193, 50-65). The viral genome encodes 6 proteins: the nucleocapsid (N) protein, the phosphoprotein (P), the matrix (M) protein, the fusion (F) protein, the hemagglutinin (H) protein, and the large (L) protein. The genes are arranged in the genomic RNA in the order (3′-5′): N, P, M, F, H, and L. Each protein is translated from a unique mRNA transcribed from the negative-strand RNA template. The H and F proteins are both glycoproteins, and localized in the viral envelope. The protein precursor (F0) undergoes a posttranslational cleavage, yielding a F1 subunit protein (Cherpillod et al, 2004, Arch. Virol. 149, 1971-1983).

### SUMMARY OF THE INVENTION

The present invention generally relates to the use of recombinant Parapoxviruses, and in particular the use of Parapoxvirus ovis (PPVO), for mediating a rapid innate immune response, as well as a long-lasting foreign gene-specific immunity against canine distemper virus.

In one embodiment, a recombinant parapoxvirus comprises heterologous DNA derived from a canine distemper virus. In one embodiment, the recombinant parapoxvirus comprises Parapoxvirus ovis strain D1701. In one embodiment, the recombinant parapoxvirus comprises Parapoxvirus ovis strain D1701-V. In one embodiment, the recombinant parapoxvirus is Parapoxvirus ovis D1701-V-CDV-H. In yet another embodiment, the recombinant parapoxvirus is Parapoxvirus ovis D1701-V-CDV-F. In one embodiment, the recombinant parapoxvirus comprises the gene encoding the H protein of the canine distemper virus, or fragments thereof. In one embodiment, the recombinant parapoxvirus comprises the gene encoding the F protein of the canine distemper virus, or fragments thereof. In one embodiment, the recombinant parapoxvirus comprises SEQ ID NO: 1, or a polynucleotide molecule having at least 98% identity to SEQ ID NO: 1. In one embodiment, the recombinant parapoxvirus comprises SEQ ID NO: 2, or a polynucleotide molecule having at least 98% identity to SEQ ID NO: 2. In one embodiment, the heterologous DNA is inserted within the HindIII fragment H/H of Parapoxvirus ovis strain D1701. In another embodiment, the heterologous DNA is inserted within the VEGF coding sequence or adjacent non-coding sequences within the HindIII fragment H/H of Parapoxvirus ovis strain D1701.

The present invention embraces methods of preparing a recombinant parapoxvirus comprising inserting heterologous DNA into the genome of the parapoxvirus. In one embodiment, the method comprises the use of Parapoxvirus ovis. In one embodiment, the method comprises the use of Parapoxvirus ovis strain D1701. In one embodiment, the method comprises the use of Parapoxvirus ovis strain D 1701-V. In one embodiment, the method comprises the preparation of Parapoxvirus ovis D1701-V-CDV-H. In one embodiment, the method comprises the preparation of Parapoxvirus ovis D1701-V-CDV-F. In one embodiment, the heterologous DNA used in the method comprises the gene encoding the H protein of the canine distemper virus, or fragments thereof. In one embodiment, the heterologous DNA used in the method comprises SEQ ID NO: 1, or a polynucleotide molecule having at least 98% identity to SEQ ID NO: 1. In one embodiment, the heterologous DNA comprises SEQ ID NO: 2, or a polynucleotide molecule having at least 98% identity to SEQ ID NO: 2.

The present invention embraces a vaccine or an immunogenic composition comprising a recombinant parapoxvirus comprising heterologous DNA derived from a canine distemper virus and a carrier. In one embodiment, the recombinant parapoxvirus comprises Parapoxvirus ovis. In one embodiment, the recombinant parapoxvirus comprises Parapoxvirus ovis strain D1701. In another embodiment, the recombinant parapoxvirus comprises Parapoxvirus ovis strain D 1701-V. In one embodiment, the recombinant parapoxvirus is Parapoxvirus ovis D1701-V-CDV-H. In one embodiment, the recombinant parapoxvirus is Parapoxvirus ovis D1701-V-CDV-F. In one embodiment, the heterologous DNA comprises the gene encoding the H protein of the canine distemper virus, or fragments thereof. In one embodiment, the heterologous DNA comprises the gene encoding the F protein of the canine distemper virus, or fragments thereof. In one embodiment, the heterologous DNA comprises SEQ ID NO: 1, or a polynucleotide molecule having at least 98% identity to SEQ ID NO: 1. In one embodiment, the heterologous DNA comprises SEQ ID NO: 2 or a polynucleotide molecule having at least 98% identity to SEQ ID NO: 2.

The present invention embraces a method of preparing a vaccine or an immunogenic composition comprising combining a recombinant parapoxvirus comprising heterologous DNA derived from a canine distemper virus and a carrier. In one embodiment, the recombinant parapoxvirus comprises Parapoxvirus ovis. In one embodiment, the recombinant parapoxvirus comprises Parapoxvirus ovis strain D1701. In another embodiment, the recombinant parapoxvirus comprises Parapoxvirus ovis strain D 1701-V. In one embodiment, the recombinant parapoxvirus is Parapoxvirus ovis D1701-V-CDV-H. In one embodiment, the recombinant parapoxvirus is Parapoxvirus ovis D1701-V-CDV-F. In one embodiment, the heterologous DNA comprises the gene encoding the H protein of the canine distemper virus, or fragments thereof. In one embodiment, the heterologous DNA comprises the gene encoding the F protein of the canine distemper virus, or fragments thereof. In one embodiment, the heterologous DNA comprises SEQ ID NO: 1, or a polynucleotide molecule having at least 98% identity to SEQ ID NO: 1. In one embodiment, the heterologous DNA comprises SEQ ID NO: 2, or a polynucleotide molecule having at least 98% identity to SEQ ID NO: 2.

The present invention embraces a method of inducing in an animal subject an immune response against canine distemper virus comprising administering to said animal a therapeutically effective amount of a vaccine or an immunogenic composition comprising the recombinant parapoxvirus comprising heterologous DNA derived from a canine distemper virus and a carrier. In one embodiment, the recombinant parapoxvirus comprises Parapoxvirus ovis. In one embodiment, the recombinant parapoxvirus comprises Parapoxvirus ovis strain D1701. In another embodiment, the recombinant parapoxvirus is Parapoxvirus ovis strain D 1701-V. In one embodiment, the recombinant parapoxvirus is Parapoxvirus ovis D1701-V-CDV-H. In one embodiment, the recombinant parapoxvirus is Parapoxvirus ovis D1701-V-CDV-F. In one embodiment, the heterologous DNA comprises the gene encoding the H protein of the canine distemper virus, or fragments thereof. In one embodiment, the heterologous DNA comprises the gene encoding the F protein of the canine distemper virus, or fragments thereof. In one embodiment, the heterologous DNA comprises SEQ ID NO: 1, or a polynucleotide molecule having at least 98% identity to SEQ ID NO: 1. In one embodiment, the heterologous DNA comprises SEQ ID NO: 2, or a polynucleotide molecule having at least 98% identity to SEQ ID NO: 2. In one embodiment, the immune response is the induction of CDV-specific antibodies. In one embodiment, an anti-H protein-specific protective immune response is induced, in one embodiment, anti-F protein-specific protective immune response is induced. In another embodiment, the immune response is the induction of anti-H protein serum antibodies. In another embodiment, the immune response is the induction of anti-F protein serum antibodies.

The present invention embraces a method of vaccinating an animal subject against canine distemper disease, comprising administering to said animal a therapeutically effective amount of a vaccine or an immunogenic composition comprising the recombinant parapoxvirus comprising heterologous DNA derived from a canine distemper virus and a carrier. In one embodiment, the recombinant parapoxvirus comprises Parapoxvirus ovis. In one embodiment, the recombinant parapoxvirus comprises Parapoxvirus ovis strain D1701. In another embodiment, the recombinant parapoxvirus comprises Parapoxvirus ovis strain D 1701-V. In one embodiment, the recombinant parapoxvirus is Parapoxvirus ovis D1701-V-CDV-H. In one embodiment, the recombinant parapoxvirus is Parapoxvirus ovis D1701-V-CDV-F. In one embodiment, the heterologous DNA comprises the gene encoding the H protein of the canine distemper virus, or fragments thereof. In one embodiment, the heterologous DNA comprises the gene encoding the F protein of the canine distemper virus, or fragments thereof. In one embodiment, the heterologous DNA comprises SEQ ID NO: 1, or a polynucleotide molecule having at least 98% identity to SEQ ID NO: 1. In one embodiment, the heterologous DNA comprises SEQ ID NO: 2, or a polynucleotide molecule having at least 98% identity to SEQ ID NO: 2.

The present invention embraces a method of treating an animal subject against canine distemper disease, comprising administering to said animal a therapeutically effective amount of a recombinant parapoxvirus comprising heterologous DNA derived from a canine distemper virus and a carrier. In one embodiment, the recombinant parapoxvirus comprises Parapoxvirus ovis. In one embodiment, the recombinant parapoxvirus comprises Parapoxvirus ovis strain D1701. In another embodiment, the recombinant parapoxvirus comprises Parapoxvirus ovis strain D 1701-V. In one embodiment, the recombinant parapoxvirus is Parapoxvirus ovis D1701-V-CDV-H. In one embodiment, the recombinant parapoxvirus is Parapoxvirus ovis D1701-V-CDV-F. In one embodiment, the heterologous DNA comprises the gene encoding the H protein of the canine distemper virus, or fragments thereof. In one embodiment, the heterologous DNA comprises the gene encoding the F protein of the canine distemper virus, or fragments thereof. In one embodiment, the heterologous DNA comprises SEQ ID NO: 1, or a polynucleotide molecule having at least 98% identity to SEQ ID NO: 1. In one embodiment, the heterologous DNA comprises SEQ ID NO: 2 or a polynucleotide molecule having at least 98% identity to SEQ ID NO: 2.

The present invention embraces a use in the preparation of a medicament for treating an animal against canine distemper disease of a recombinant parapoxvirus comprising heterologous DNA derived from a canine distemper virus. In one embodiment, the recombinant parapoxvirus comprises Parapoxvirus ovis strain D1701. In one embodiment, the recombinant parapoxvirus comprises Parapoxvirus ovis strain D1701-V. In one embodiment, the recombinant parapoxvirus is Parapoxvirus ovis D1701-V-CDV-H. In yet another embodiment, the recombinant parapoxvirus is Parapoxvirus ovis D1701-V-CDV-F. In one embodiment, the recombinant parapoxvirus comprises the gene encoding the H protein of the canine distemper virus, or fragments thereof. In one embodiment, the recombinant parapoxvirus comprises the gene encoding the F protein of the canine distemper virus, or fragments thereof. In one embodiment, the recombinant parapoxvirus comprises SEQ ID NO: 1, or a polynucleotide molecule having at least 98% identity to SEQ ID NO: 1. In one embodiment, the recombinant parapoxvirus comprises SEQ ID NO: 2, or a polynucleotide molecule having at least 98% identity to SEQ ID NO: 2. In one embodiment, the heterologous DNA is inserted within the HindIII fragment H/H of Parapoxvirus ovis strain D1701. In another embodiment, the heterologous DNA is inserted within the VEGF coding sequence or adjacent non-coding sequences within the HindIII fragment H/H of Parapoxvirus ovis strain D1701.

The present invention embraces a use of a parapoxvirus in the preparation of a medicament for vaccinating an animal against canine distemper disease. In one embodiment, a recombinant parapoxvirus comprising heterologous DNA derived from a canine distemper virus is provided. In one embodiment, the recombinant parapoxvirus comprises Parapoxvirus ovis strain D1701. In one embodiment, the recombinant parapoxvirus comprises Parapoxvirus ovis strain D1701-V. In one embodiment, the recombinant parapoxvirus is Parapoxvirus ovis D1701-V-CDV-H. In one embodiment, the recombinant parapoxvirus is Parapoxvirus ovis D1701-V-CDV-F. In another embodiment, the heterologous DNA comprises the gene encoding for the H protein of CDV. In another embodiment, the heterologous DNA comprises the gene encoding for the F protein of CDV. In one embodiment, the heterologous DNA comprises SEQ ID NO: 1, or a polynucleotide molecule having at least 98% identity to SEQ ID NO: 1. In one embodiment, the heterologous DNA comprises SEQ ID NO: 2, or a polynucleotide molecule having at least 98% identity to SEQ ID NO: 2.

The present invention provides methods of determining the origin of a Parapoxvirus present in an animal subject. The Parapoxviruses described herein can be distinguished from wild-type strains in both their genomic composition and proteins expressed. Such distinction allows for discrimination between vaccinated and infected animals. The recombinant Parapoxvirus ovis D1701-V-CDV-H can be used in a DIVA assay. The recombinant Parapoxvirus ovis D1701-V-CDV-F can be used in a DIVA assay.

These and other embodiments are disclosed and encompassed by the following detailed description.

### BRIEF DESCRIPTION OF THE DRAWING(S)

A better understanding of the present invention will be gained by referring to the accompanying drawings in which:

FIG. 1: Construction of plasmids pdV-CDV-H and pdV-CDV-F. The CDV H, as well as the CDV F gene, were cloned as BamHI-KpnI fragments into the transfer plasmid pdV-Rec1 to obtain plasmids pdV-CDV-H (1A) or pdV-CDV-F (1B).

FIG. 2: Immune peroxidase staining (IPMA) of cells infected with D1701-V-CDV-F or D1701-V-CDV-H. Vero cells were infected with the recombinant D1701-V-CDV-F (2A) or D1701-V-CDV-H (2B). Control (non-infected) Vero cells are shown in 2C. Dark stained cells demonstrate specific protein expression.

FIG. 3: Immunofluorescent staining of the CDV-F or -H protein expressed by the recombinants. Vero cells were infected with recombinant D1701-V-CDV-H for 8 hours (3A) and 12 hours (3B); or with recombinant D1701-V-CDV-H for 24 hours (3C). Specific staining was achieved with rabbit anti-CDV-H antibody (diluted 1:2000) or with rabbit anti-CDV-F antibody (diluted 1:200), and anti-rabbit-Alexa-555, diluted 1:2000. For negative control, non-infected cells were used (D). Specifically stained cells are indicated by arrows.

FIG. 4: Western blot analysis of the F-gene of CDV expressed in ORFV recombinant-infected cells. Protein lysates were prepared from non-infected Vero cells (ni), cells infected with recombinant D701-V-CDV-F (MOI=3.0) for 4 to 72 hours (4, 8, 24, 48 and 72 post-infection), and from CDV Ondersteport (reference strain) infected cells (CDV control).

FIG. 5: Single step growth curve (SSGC) of D1701-V-CDV-H. Vero cells were infected (MOI=3.0) with the parental virus D1701-V (open circles) or with the recombinant D1701-V-CDV-H (closed circles).

FIG. 6. Detection of CDV-H specific antibodies by immunofluorescence. Serum (1:1000 dilution) of mice immunized i.m. with recombinant D1701-V-CDV-H (10⁷ pfu), FIGS. 6 A-C show Vero cells infected with CDV strain Onderstepoort. Arrows demonstrate specific reaction with mouse antiserum. FIG. 6 D shows non-infected Vero cells.

FIG. 7: Sequence of the cloned H gene fragment. The coding region of the canine distemper virus H protein (SEQ ID NO: 1; 1824 nt), plus 20 nt on the 5′-end and 21 nt on the 3′-end as linker sequences for restriction enzyme cloning and analysis (BamHI and KpnI). The ATG start codon is underlined.

FIG. 8: Sequence of the cloned F gene fragment. The coding region of the canine distemper virus F protein (SEC) ID NO: 2; 1989 nt), plus 19 nt on the 5′-end and 16 nt on the 3′-end as linker sequences for restriction enzyme cloning and analysis (BamHI and KpnI). The ATG start codon is underlined.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular.

The following definitions may be applied to terms employed in the description of embodiments of the invention. They supersede any contradictory definitions contained in each individual reference incorporated herein by reference.

“About” or “approximately,” when used in connection with a measurable numerical variable, refers to the indicated value of the variable and to all values of the variable that are within the experimental error of the indicated value (e.g., within the 95% confidence interval for the mean) or within 10 percent of the indicated value, whichever is greater, unless about is used in reference to time intervals in weeks where “about 3 weeks,” is 17 to 25 days, and about 2 to about 4 weeks is 10 to 40 days.

The terms “animal” and “subject”, as used herein, includes any animal that is susceptible to canine distemper infections, including mammals, both domesticated and wild.

“Antibody”, as used herein, is any polypeptide comprising an antigen-binding site regardless of the source, method of production, or other characteristics. It refers to an immunoglobulin molecule or a fragment thereof that specifically binds to an antigen as the result of an immune response to that antigen. Immunoglobulins are serum proteins composed of “light” and “heavy” polypeptide chains having “constant” and “variable” regions and are divided into classes (e.g., IgA, IgD, IgE, IgG, and IgM) based on the composition of the constant regions. An antibody that is “specific” for a given antigen indicates that the variable regions of the antibody recognize and bind a specific antigen exclusively. The term includes, but is not limited to: a polyclonal antibody, a monoclonal antibody, a monospecific antibody, polyspecific antibody, humanized antibody, a tetrameric antibody, a tetravalent antibody, a multispecific antibody, a single chain antibody, a domain-specific antibody, a single domain antibody, a domain-deleted antibody, a fusion protein, an ScFc fusion protein, a single-chain antibody, chimeric antibody, synthetic antibody, recombinant antibody, hybrid antibody, mutated antibody, and CDR-grafted antibodies. Antibodies can be intact immunoglobulins derived from natural sources or from recombinant sources, or can be immunoreactive portions of intact immunoglobulins. An “antibody” can be converted to an antigen-binding protein, which includes but is not limited to antibody fragments which include but are not limited to: Fab, F(ab′)₂, an Fab′ fragment, an Fv fragment, a single-chain Fv (ScFv) fragment, an Fd fragment, a dAb fragment, diabodies, a CDR3 peptide, a constrained FR3-CDR3-FR4 peptide, a nanobody, a bivalent nanobody, a small modular immunopharmaceutical (SMIPs), and a minibody and any of above mentioned fragments and their chemically or genetically manipulated counterparts, as well as other antibody fragments that retain antigen-binding function. Typically, such fragments would comprise an antigen-binding domain. As will be recognized by those of skill in the art, any of such molecules may be engineered (for example “germlined”) to decrease its immunogenicity, increase its affinity, alter its specificity, or for other purposes.

“Antigen” or “immunogen”, as used herein, refers to a molecule that contains one or more epitopes (linear, conformational or both) that upon exposure to a subject will induce an immune response that is specific for that antigen. The term antigen refers to subunit antigens—antigens separate and discrete from a whole organism with which the antigen is associated in nature—as well as killed, attenuated or inactivated bacteria, viruses, fungi, parasites or other microbes. The term antigen also refers to antibodies, such as anti-idiotype antibodies or fragments thereof, and to synthetic peptide mimotopes that can mimic an antigen or antigenic determinant (epitope). The term antigen also refers to an oligonucleotide or polynucleotide that expresses an antigen or antigenic determinant in vivo, such as in DNA immunization applications.

“Antigenicity”, as used herein, refers to the capability of a protein or polypeptide to be immunospecifically bound by an antibody raised against the protein or polypeptide.

“Buffer” means a chemical system that prevents change in the concentration of another chemical substance. Proton donor and acceptor systems serve as buffers preventing marked changes in hydrogen ion concentration (pH). A further example of a buffer is a solution containing a mixture of a weak acid and its salt (conjugate base) or a weak base and its salt (conjugate acid).

“Canine”, as used herein, includes what is commonly called the dog, but includes other members of the family Canidae.

The term “canine distemper virus” refers to a member of the Morbillivirus genus, in the Paramyxoviridae family, in the order Mononegavirales.

The term “cell line” or “host cell”, as used herein, means a prokaryotic or eukaryotic cell in which a virus can replicate or be maintained.

“Cellular immune response” or “cell mediated immune response” is one mediated by T-lymphocytes or other white blood cells or both, and includes the production of cytokines, chemokines and similar molecules produced by activated T-cells, white blood cells, or both.

“Conservative substitution” is defined in the art and known to one skilled in the art, and is recognized to classify residues according to their related physical properties.

The term “culture”, as used herein, means a population of cells or microorganisms growing in the absence of other species or types.

The term “DIVA” as used herein means a vaccine or an immunogenic composition which is able to differentiate infected from vaccinated animals.

“Dose” refers to a vaccine or immunogenic composition given to a subject. A “first dose” or “priming dose” refers to the dose of such a composition given on Day 0. A “second dose” or a “third dose” or an “annual dose” refers to an amount of such composition given subsequent to the first dose, which may or may not be the same vaccine or immunogenic composition as the first dose.

An “epitope” is the specific site of the antigen which binds to a T-cell receptor or specific antibody, and typically comprises from about 3 amino acid residues to about 20 amino acid residues.

“Excipient”, as used herein, refers to any component of a vaccine or immunogenic composition that is not an antigen.

“Fragment” refers to a truncated portion of a protein or gene. “Functional fragment” and “biologically active fragment” refer to a fragment that retains the biological properties of the full length protein or gene. An “immunogenically active fragment” refers to a fragment that elicits an immune response.

The term “F protein”, as used herein, refers to the fusion protein of canine distemper virus.

The term “H” protein, as used herein, refers to the hemagglutinin glycoprotein of canine distemper virus.

The term “heterologous”, as used herein, means derived from a different species or strain.

The term “homologous”, as used herein, means derived from the same species or strain.

“Homology” or “percent homology” refers to the percentage of nucleotide or amino acid residues in the candidate sequence that are identical with the residues in the comparator sequence(s) after aligning the sequences and introducing gaps, if necessary, to achieve the maximum percent sequence homology, and also considering any conservative substitutions as part of the sequence homology.

“Homologs” or “Species homologs” include genes found in two or more different species which possess substantial polynucleotide sequence homology and possess the same, or similar, biological functions and/or properties. Preferably polynucleotide sequences which represent species homologs will hybridize under moderately stringent conditions, as described herein by example, and possess the same or similar biological activities and or properties. In another aspect, polynucleotides representing species homologs will share greater than about 60% sequence homology, greater than about 70% sequence homology, greater than about 80% sequence homology, greater than about 90% sequence homology, greater than about 95% sequence homology, greater than about 96% sequence homology, greater than about 97% sequence homology, greater than about 98% sequence homology, or greater than about 99% sequence homology.

“Humoral immune response” refers to one that is at least in part mediated by antibodies.

“Identity” or “percent identity” refers to the percentage of nucleotides or amino acids in the candidate sequence that are identical with the residues in the comparator sequence after aligning both sequences and introducing gaps, if necessary, to achieve the maximum percent sequence identity, and not considering any conservative substitutions as part of the sequence identity.

“Immune response” in a subject refers to the development of a humoral immune response, a cellular immune response, or a humoral and a cellular immune response to an antigen. The immune response may be sufficient for diagnostic purposes or other testing, or may be adequate to prevent signs or symptoms of disease, including adverse health effects or complications thereof, caused by infection with a disease agent. Immune responses can usually be determined using standard immunoassays and neutralization assays, which are known in the art.

“Immunogenic” or “immunogenicity”, as used herein, refers to the capability to elicit an immune response directed specifically against an antigen.

The terms “immunogenic composition,” or “immunologically effective amount,” or “amount effective to produce an immune response,” as used herein, refer to a composition or antigen capable of being recognized by the immune system, resulting in the generation of a specific immune response (i.e., has immunogenic activity) when administered alone or with a pharmaceutically acceptable carrier, to an animal.

“Intranasal” administration, as used herein, refers to the introduction of a substance, such as a vaccine or immunogenic composition, into a subject's body through or by way of the nose, and involves transport of the substance primarily through the nasal mucosa.

“Isolated”, as used herein, means removed from its naturally occurring environment, either alone or in a heterologous host cell, or chromosome or vector (e.g., plasmid, phage, etc.). An isolated microorganism refers to a composition in which the organism is substantially free of other microorganisms, e.g., in a culture, such as when separated from it naturally occurring environment. “Isolated,” when used to describe any particularly defined substance, such as a polynucleotide or a polypeptide, refers to the substance that is separate from the original cellular environment in which the substance—such as a polypeptide or nucleic acid—is normally found. As used herein therefore, by way of example only, a recombinant cell line constructed with a polynucleotide of the invention makes use of the “isolated” nucleic acid. Alternatively, if a particular protein or a specific immunogenic fragment is claimed or used as a vaccine or immunogenic composition, it would be considered to be isolated because it had been identified, separated and to some extent purified as compared to how it may exist in nature. If the protein or a specific immunogenic fragment thereof is produced in a recombinant bacterium or eukaryote expression vector that produces the antigen, it is considered to exist as an isolated protein or nucleic acid. For example, a recombinant cell line constructed with a polynucleotide makes use of an “isolated” nucleic acid.

“Medicinal agent” refers to any agent which is useful in the prevention, cure, or improvement of disease, or the prevention of some physiological condition or occurrence.

The term “multiplicity of infection” (MOI) refers to a ratio of the number of organisms per cell, which details how much inoculum is going to be used in a given infection.

“Monoclonal antibody”, as used herein, refers to antibodies produced by a single line of hybridoma cells, all directed towards one epitope on a particular antigen. The antigen used to make the monoclonal antibody can be provided as an isolated protein of the pathogen or the whole pathogen. A “hybridoma” is a clonal cell line that consists of hybrid cells formed by the fusion of a myeloma cell and a specific antibody-producing cell. In general, monoclonal antibodies are of mouse origin. However, monoclonal antibody also refers to a clonal population of an antibody made against a particular epitope of an antigen produced by phage display technology, or method that is equivalent to phage display, or hybrid cells of non-mouse origin.

“Oral” or “peroral” administration, as used herein, refers to the introduction of a substance, such as a vaccine or immunogenic composition, into a subject's body through or by way of the mouth and involves swallowing or transport through the oral mucosa (e.g., sublingual or buccal absorption) or both. Intratracheal is also a means of oral or peroral administration.

“Oronasal” administration, as used herein, refers to the introduction of a substance, such as an immunogenic composition or vaccine, into a subject's body through or by way of the nose and the mouth, as would occur, for example, by placing one or more droplets in the nose. Oronasal administration involves transport processes associated with oral and intranasal administration.

The terms “parapoxvirus”, “parapoxvirus strains”, as used herein, refer to viruses belonging to the family Poxviridae and the genus Parapoxvirus.

The terms “Parapoxvirus ovis” and “ORFV”, as used herein, refer to viruses belonging to the family Poxviridae, the genus Parapoxvirus, and the species Parapoxvirus ovis. These viruses are also called ecthyma contagiosum virus, contagious pustular dermatitis virus, or orf virus. They possess a unique spiral coat that distinguishes them from the other poxviruses.

The term “Parapoxvirus ovis strain D1701” refers to the virus as described in U.S. Pat. No. 6,365,393, which is incorporated herein by reference. “Parapoxvirus ovis strain D1701-V” refers to Parapoxvirus ovis strain D1701 adapted to the simian cell line Vero.

“Parenteral administration” refers to the introduction of a substance, such as a vaccine or immunogenic composition, into a subject's body through or by way of a route that does not include the digestive tract. Parenteral administration includes, but is not limited to, subcutaneous, intramuscular, transcutaneous, intradermal, intraperitoneal, intraocular, and intravenous administration.

The term “pathogen” or “pathogenic microorganism”, as used herein, means a microorganism—for example a canine distemper virus—which is capable of inducing or causing a disease, illness, or abnormal state in its host animal.

“Pharmaceutically acceptable” refers to substances, which are within the scope of sound medical judgment, suitable for use in contact with the tissues of subjects without undue toxicity, irritation, allergic response, and the like, commensurate with a reasonable benefit-to-risk ratio, and effective for their intended use.

“Polyclonal antibody”, as used herein, refers to a mixed population of antibodies made against a particular pathogen or antigen. In general, the population contains a variety of antibody groups, each group directed towards a particular epitope of the pathogen or antigen. To make polyclonal antibodies, the whole pathogen, or an isolated antigen, is introduced by inoculation or infection into a host, which induces the host to make antibodies against the pathogen or antigen.

The term “poxvirus”, as used herein, refers to viruses belonging to the family Poxviridae. These viruses are oval, quite large, double-stranded DNA viruses.

The term “polynucleotide”, as used herein, means an organic polymer molecule composed of nucleotide monomers covalently bonded in a chain. DNA (deoxyribonucleic acid) and RNA (ribonucleic acid) are examples of polynucleotides with distinct biological function.

The term “polypeptide”, as used herein, means an organic polymer molecule composed of two or more amino acids bonded in a chain.

The terms “prevent”, “preventing” or “prevention”, and the like, as used herein, mean to inhibit the replication of a microorganism, to inhibit transmission of a microorganism, or to inhibit a microorganism from establishing itself in its host. These terms and the like as used herein can also mean to inhibit or block or alleviate one or more signs or symptoms of infection.

“Protection”, “protecting”, “protective immunity”, and the like, as used herein with respect to a vaccine or immunogenic composition, means that the vaccine or composition prevents or reduces the symptoms of the disease caused by the organism from which the antigen(s) used in the vaccine or immunogenic composition is derived. The terms “protection” and “protecting” and the like, also mean that the vaccine or immunogenic composition can be used to therapeutically treat the disease or one of more symptoms of the disease that already exists in a subject.

“Recombinantly prepared PPV” or “Recombinant PPV” are PPV having insertions and/or deletions in their genome. The insertions and deletions are prepared using molecular biological methods.

The terms “specific binding,” “specifically binds,” and the like, are defined as two or more molecules that form a complex that is measurable under physiologic or assay conditions and is selective. An antibody or other inhibitor is said to “specifically bind” to a protein if, under appropriately selected conditions, such binding is not substantially inhibited, while at the same time non-specific binding is inhibited. Specific binding is characterized by high affinity and is selective for the compound or protein. Nonspecific binding usually has low affinity. Binding in IgG antibodies, for example, is generally characterized by an affinity of at least about 10⁻⁷ M or higher, such as at least about 10⁻⁸ M or higher, or at least about 10⁻⁹ M or higher, or at least about 10⁻¹ or higher, or at least about 10⁻¹¹ M or higher, or at least about 10⁻¹² M or higher. The term is also applicable where, e.g., an antigen-binding domain is specific for a particular epitope that is not carried by numerous antigens, in which case the antibody carrying the antigen-binding domain will generally not bind other antigens.

“Specific immunogenic fragment”, as used herein, refers to a portion of a sequence that is recognizable by an antibody or T cell specific for that sequence.

“Substantially identical”, as used herein, refers to a degree of sequence identity of at least about 90%, at least about 95%, at least about 96%, at least about 97%, at least about 98%, or at least about 99%.

The term “therapeutically effective amount”, as used herein, means an amount of a microorganism, or a subunit antigen, or a polypeptide, or a polynucleotide, or combinations thereof, sufficient to elicit an immune response in the subject to which it is administered.

“Therapeutic agent”, as used herein, refers to any molecule, compound, virus or treatment, preferably a virus attenuated or killed, or subunit or compound, that assists in the treatment of a viral, bacterial, parasitic or fungal infection, disease or condition caused thereby.

“Therapeutically effective amount”, as used herein, refers to an amount of an antigen or vaccine or immunogenic composition that would induce an immune response in a subject (e.g., dog) receiving the antigen or vaccine or immunogenic composition which is adequate to prevent or ameliorate signs or symptoms of disease, including adverse health effects or complications thereof, caused by infection with a pathogen, such as a virus, bacterium, parasite or fungus. Humoral immunity or cell-mediated immunity, or both humoral and cell-mediated immunity, can be induced. The immunogenic response of an animal to an antigen, vaccine, or immunogenic composition can be evaluated indirectly through measurement of antibody titers, lymphocyte proliferation assays, or directly through monitoring signs and symptoms after challenge with the wild type strain. The protective immunity conferred by a vaccine or immunogenic composition can be evaluated by measuring reduction of challenge organism shed, and/or reduction in clinical signs, such as mortality, morbidity, temperature, and overall physical condition, health, and performance of the subject. The amount of a vaccine or immunogenic composition that is therapeutically effective can vary, depending on the particular immunogen used, or the condition of the subject, and can be determined by one skilled in the art.

The terms “treat”, “treating” or “treatment”, and the like, as used herein, mean to prevent, reduce, or eliminate an infection by a microorganism. These terms and the like can also mean to reduce the replication of a microorganism, to reduce the transmission of a microorganism, to reduce the ability of a microorganism to establish itself in its host, or to prevent a microorganism from establishing itself in its host. These terms and the like as used herein can also mean to reduce, ameliorate, or eliminate one or more signs or symptoms of infection by a microorganism, or accelerate the recovery from infection by a microorganism.

The terms “vaccinate” and “vaccinating” and the like, as used herein, mean to administer to an animal a vaccine or immunogenic composition.

The terms “vaccine” and “vaccine composition,” as used herein, mean a composition comprising a virus or bacteria, either modified live, attenuated, or killed, or a subunit vaccine, or any combination of the aforementioned, which prevents or reduces an infection, or which prevents or reduces one or more signs or symptoms of infection. The protective effects of a vaccine against a pathogen are normally achieved by inducing in the subject an immune response. Generally speaking, abolished or reduced incidences of infection, amelioration of the signs or symptoms, or accelerated elimination of the microorganism from the infected subjects are indicative of the protective effects of a vaccine composition. The vaccines described herein provide protective effects against infections caused by canine distemper virus.

The term “variant,” as used herein, refers to a derivation of a given protein and/or gene sequence, wherein the derived sequence is essentially the same as the given sequence, but for mutational differences. Said differences may be naturally-occurring, or synthetically- or genetically-generated.

A “vector” or a “vector virus” is a PPV which is suitable for the insertion of heterologous DNA, which can transport the inserted DNA into cells or organisms, and which, where appropriate, enables the heterologous DNA to be expressed.

The term “veterinarily acceptable” as used herein refers to substances, which are within the scope of sound medical judgment, suitable for use in contact with the tissues of animals without undue toxicity, irritation, allergic response, and the like, commensurate with a reasonable benefit-to-risk ratio, and effective for their intended use.

The term “veterinarily acceptable carrier” as used herein refers to a carrier medium that does not interfere with the effectiveness of the biological activity of the active ingredient, and is not toxic to the veterinary subject to whom it is administered.

### Viruses, Immunogenic Compositions, and Vaccines

The present invention embraces the use of parapoxviruses for the preparation of a recombinant parapoxvirus comprising heterologous DNA derived from a canine distemper virus.

In one embodiment, Parapoxvirus ovis (PPVO) for the preparation of a recombinant parapoxvirus comprising heterologous DNA derived from a canine distemper virus is used. In another embodiment, the Parapoxvirus ovis strain D1701 is used. This strain is described in U.S. Pat. No. 6,365,393; Rziha et al., 2000, J. Biotechnol., 83, 137-145; and Cottone, et al., 1998, Virus Research, 56, 53-67. In a further embodiment, the Parapoxvirus ovis strain D1701-V is used.

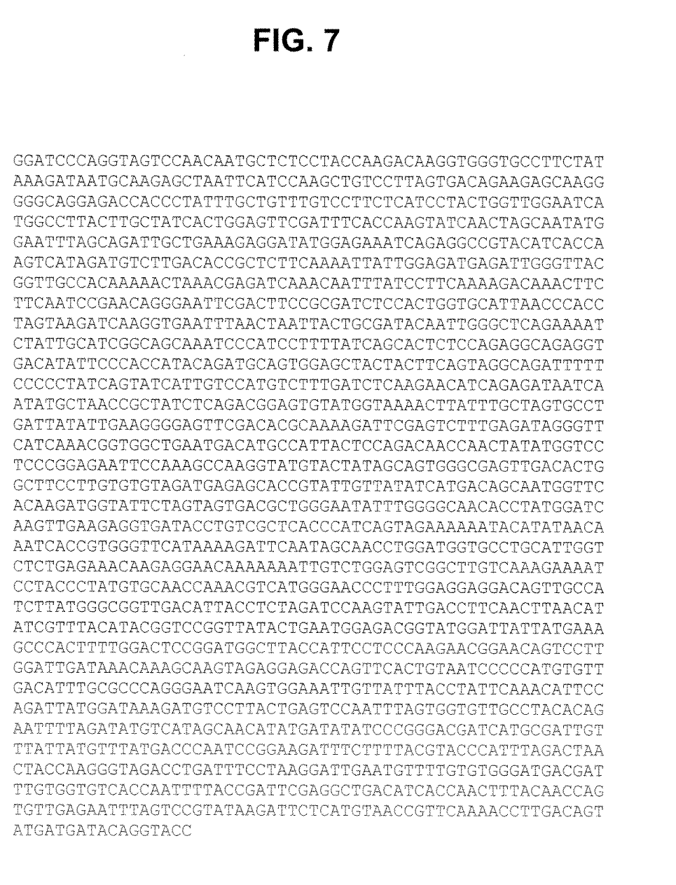

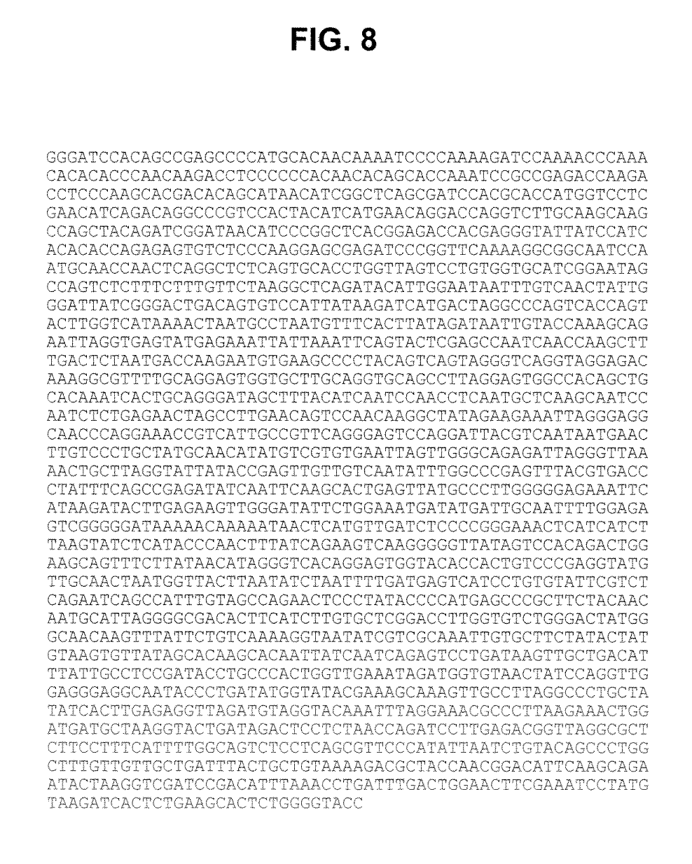

The genetic sequence inserted into the parapoxvirus includes heterologous DNA derived from a canine distemper virus. In one embodiment, the heterologous DNA comprises the genes encoding the H protein of the canine distemper virus, or fragments thereof. In one embodiment, the heterologous DNA comprises SEQ ID NO: 1 or a polynucleotide molecule having at least 98% identity to SEQ ID NO: 1. The complete sequence of the cloned fragment containing the H gene of canine distemper virus (SEQ ID NO: 1) is shown in FIG. 7. It contains the full-length coding region (1824 nt), with linker sequences on the 5′-(20 nt) and 3′-(21 nt) ends for restriction enzyme cloning and analysis (BamHI and KpnI). In one embodiment, the heterologous DNA comprises the gene encoding the F protein of the canine distemper virus, or fragments thereof. In one embodiment, the heterologous DNA comprises SEQ ID NO: 2, or a polynucleotide molecule having at least 98% identity to SEQ ID NO: 2. The complete sequence of the cloned fragment containing the F gene of canine distemper virus (SEQ ID NO: 2) is shown in FIG. 8. It contains the full-length coding region (1989 nt), with linker sequences on the (19 nt) and 3′-(16 nt) ends for restriction enzyme cloning and analysis (BamHI and KpnI).

Knowledge of the sequence of a polynucleotide makes readily available every possible fragment of that polynucleotide. The invention therefore provides for fragments of the H protein. The invention therefore also provides for fragments of the F protein. In one embodiment, functional fragments are provided for. In another embodiment, biologically active fragments are provided for. Fragments can be purified by conventional methods, such as for example by filtration or chromatography. Fragments can be produced by recombination by methods known to one skilled in the arts.

In preparing the recombinant parapoxvirus, the heterologous DNA is inserted within the HindIII fragment H/H of Parapoxvirus ovis strain D1701. In another embodiment, the heterologous DNA is inserted in within the VEGF coding sequence or adjacent non-coding sequences within the HindIII fragment H/H of Parapoxvirus ovis strain D1701. The methods used to insert the heterologous DNA into the parapoxvirus are standard and known to one skilled in the art. They are described in U.S. Pat. No. 6,365,393.

In one embodiment, the recombinant parapoxvirus comprising heterologous DNA derived from a canine distemper virus is Parapoxvirus ovis D1701-V-CDV-H or Parapoxvirus ovis D1701-V-CDV-F. These viruses are being deposited at the European Collection of Cell Cultures (ECACC), Porton Down, Salisbury, Wiltshire SP4 CMG, UK, which is a part of the Health Protection Agency Culture Collections (HPA Culture Collections), in compliance with the Budapest Treaty on the International Recognition of the Deposit of Microorganisms for the Purposes of Patent Procedure.

The sequence of the plasmid pdV-CDV-H (7,975 nt) is SEQ ID NO: 3, which is shown in the sequence listing.

The sequence of the plasmid pdV-CDV-F (8,134 nt) is SEQ ID NO: 4, which is shown in the sequence listing.

The invention also embraces polynucleotide sequences that have at least about 99%, at least about 98%, at least about 97%, at least about 96%, at least about 95%, at least about 93%, at least about 90%, at least about 85%, at least about 80%, at least about 75%, at least about 70%, at least about 65%, at least about 60%, at least about 55%, and at least about 50% identity and/or homology to the sequences described herein.

The invention also embraces polynucleotide sequences which hybridize under moderately to highly stringent conditions to the non-coding strand, or complement, of any one of the SEQ ID NOs described herein, and species homologs thereof. Exemplary high stringency conditions include a final wash in buffer comprising 0.2×SSC/0.1% SDS, at 65° C. to 75° C., while exemplary moderate stringency conditions include a final wash in buffer comprising 2×SSC/0.1% SDS, at 35° C. to 45° C. It is understood in the art that conditions of equivalent stringency can be achieved through variation of temperature and buffer, or salt concentration as described in Ausubel, at al. (Eds.). Protocols in Molecular Biology, John Wiley & Sons (1994), pp. 6.03 to 6.4.10.

The recombinant PPV can be propagated in cells, cell lines and host cells. Said cells, cell lines, or host cells may be for example, but are not limited to, mammalian cells and non-mammalian cells. Cells, cell lines, and host cells in which the PPV can be propagated are readily known and accessible to those of ordinary skill in the art. In one embodiment Vero cells are used. In other embodiments, bovine kidney or ovine testis cells are used.

The recombinant PPV can be further attenuated or inactivated prior to use in an immunogenic composition or vaccine. Methods of attenuation and inactivation are well known to those skilled in the art. Methods for attenuation include, but are not limited to, serial passage in cell culture on a suitable cell line, ultraviolet irradiation, and chemical mutagenesis. Methods for inactivation include, but are not limited to, treatment with formalin, betapropriolactone (BPL) or binary ethyleneimine (BEI), or other methods known to those skilled in the art.

Inactivation by formalin can be performed by mixing the virus suspension with 37% formaldehyde to a final formaldehyde concentration of 0.05%. The virus-formaldehyde mixture is mixed by constant stirring for approximately 24 hours at room temperature. The inactivated virus mixture is then tested for residual live virus by assaying for growth on a suitable cell line.

Inactivation by BEI can be performed by mixing the virus suspension of the present invention with 0.1 M BEI (2-bromo-ethylamine in 0.175 N NaOH) to a final BEI concentration of 1 mM. The virus-BEI mixture is mixed by constant stirring for approximately 48 hours at room temperature, followed by the addition of 1.0 M sodium thiosulfate to a final concentration of 0.1 mM. Mixing is continued for an additional two hours. The inactivated virus mixture is tested for residual live virus by assaying for growth on a suitable cell line.

The recombinant PPV can be used in immunogenic compositions and vaccines.

The immunogenic compositions and vaccines optionally can include one or more veterinarily acceptable carriers, including liquid, semisolid, or solid diluents, that serve as pharmaceutical vehicles, excipients, or media. As used herein, a “veterinarily-acceptable carrier” includes any and all solvents, dispersion media, coatings, adjuvants, stabilizing agents, diluents, preservatives, antibacterial and antifungal agents, isotonic agents, adsorption delaying agents, and the like. Diluents can include water, saline, dextrose, ethanol, glycerol, and the like. Isotonic agents can include sodium chloride, dextrose, mannitol, sorbitol, and lactose, among others known to those skilled in the art. Stabilizers include albumin, among others known to the skilled artisan. Preservatives include merthiolate, among others known to the skilled artisan.

Adjuvants include, but are not limited to, the RIBI adjuvant system (Ribi Inc.), alum, aluminum hydroxide gel, oil-in water emulsions, water-in-oil emulsions such as, e.g., Freund's complete and incomplete adjuvants, Block co polymer (CytRx, Atlanta Ga.), SAF-M (Chiron, Emeryville Calif.), AMPHIGEN® adjuvant, saponin, Quil A, QS-21 (Cambridge Biotech Inc., Cambridge Mass.), GPI-0100 (Galenica Pharmaceuticals, Inc., Birmingham, Ala.) or other saponin fractions, monophosphoryl lipid A, Avridine lipid-amine adjuvant, heat-labile enterotoxin from E. coli (recombinant or otherwise), cholera toxin, or muramyl dipeptide, among many others known to those skilled in the art. The amounts and concentrations of adjuvants and additives useful in the context of the present invention can readily be determined by the skilled artisan. In one embodiment, the present invention contemplates immunogenic compositions and vaccines comprising from about 50 μg to about 2000 μg of adjuvant. In another embodiment adjuvant is included in an amount from about 100 μg to about 1500 μg, or from about 250 μg to about 1000 μg, or from about 350 μg to about 750 μg. In another embodiment, adjuvant is included in an amount of about 500 μg/2 ml dose of the immunogenic composition or vaccine.

The immunogenic compositions and vaccines can also include antibiotics. Such antibiotics include, but are not limited to, those from the classes of aminoglycosides, carbapenems, cephalosporins, glycopeptides, macrolides, penicillins, polypeptides, quinolones, sulfonamides, and tetracyclines. In one embodiment, the present invention contemplates immunogenic compositions and vaccines comprising from about 1 μg/ml to about 60 μg/ml of antibiotic. In another embodiment, the immunogenic compositions and vaccines comprise from about 5 μg/ml to about 55 μg/ml of antibiotic, or from about 10 μg/ml to about 50 μg/ml of antibiotic, or from about 15 μg/ml to about 45 μg/ml of antibiotic, or from about 20 μg/ml to about 40 μg/ml of antibiotic, or from about 25 μg/ml to about 35 μg/ml of antibiotic. In yet another embodiment, the immunogenic compositions and vaccines comprise less than about 30 μg/ml of antibiotic.

In addition to the recombinant PPV, immunogenic compositions and vaccines can include other antigens. Antigens can be in the form of an inactivated whole or partial preparation of the microorganism, or in the form of antigenic molecules obtained by genetic engineering techniques or chemical synthesis. Other antigens appropriate for use in accordance with the present invention include, but are not limited to, those derived from pathogenic bacteria or pathogenic viruses.

The recombinant canine distemper virus immunogenic compositions and vaccines can also optionally contain a mixture with one or more additional canine antigens such as, for example, Ehrlichia canis, canine parvovirus (CPV), canine parainfluenza virus (CPI), canine adenovirus type II (CAV-2), canine adenovirus (CAV), canine coronavirus (CCV). Leptospira icterohemorrhagiae (LI), Leptospira canicola (LC), Leptospira grippotyphosa (LG), Leptospira pomona (LP), Borrelia burgdorferi, and the like. One combination of antigens encompasses isolates of canine parvovirus, canine adenovirus and canine parainfluenza, with or without coronavirus and Leptospira (including the emerging serovars Leptospira grippotyphosa and Leptospira pomona).

Immunogenic compositions and vaccines described herein can be administered to an animal to induce an effective immune response against CDV. Accordingly, described herein are methods of stimulating an effective immune response against CDV comprising administering to an animal a therapeutically effective amount of an immunogenic composition or vaccine comprising a recombinant parapoxvirus comprising heterologous DNA derived from a canine distemper virus. In one embodiment, the method results in the induction of anti-H protein serum antibodies. In another embodiment, the method results in the induction of anti-F protein serum antibodies.

Immunogenic compositions and vaccines described herein can be administered to an animal to vaccinate the animal subject against canine distemper disease. The immunogenic compositions and vaccines can be administered to the animal to prevent or treat canine distemper disease in the animal. Accordingly, described herein are methods of vaccinating an animal against canine distemper disease, and preventing or treating canine distemper disease, comprising administering to the animal a therapeutically effective amount of an immunogenic composition or vaccine comprising a recombinant parapoxvirus comprising heterologous DNA derived from a canine distemper virus.

### Forms, Dosages, Routes of Administration

Immunogenic compositions and vaccines can be made in various forms depending upon the route of administration. For example, the immunogenic compositions and vaccines can be made in the form of sterile aqueous solutions or dispersions suitable for injectable use, or made in lyophilized forms using freeze-drying techniques. Lyophilized immunogenic compositions and vaccines are typically maintained at about 4° C., and can be reconstituted in a stabilizing solution, e.g., saline or and HEPES. Alternatively, immunogenic compositions and vaccines can be preserved by freeze drying. Immunogenic compositions and vaccines can also be made in the form of suspensions or emulsions.

Immunogenic compositions and vaccines include a therapeutically effective amount of the above-described recombinant PPV. Purified viruses can be used directly in an immunogenic composition or vaccine, or can be further attenuated, or inactivated. Typically, an immunogenic composition or vaccine contains between about 1×10² and about 1×10¹² PFU, or between about 1×10³ and about 1×10¹¹ PFU, or between about 1×10⁴ and about 1×10¹⁰ PFU, or between about 1×10⁵ and about 1×10⁹ PFU, or between about 1×10⁶ and about 1×10⁸ PFU. The precise amount of a virus in an immunogenic composition or vaccine effective to provide a protective effect can be determined by a skilled artisan.

The immunogenic compositions and vaccines generally comprise a veterinarily acceptable carrier in a volume of between about 0.5 ml and about 5 ml. In another embodiment the volume of the carrier is between about 1 ml and about 4 ml, or between about 2 ml and about 3 ml. In another embodiment, the volume of the carrier is about 1 ml, or is about 2 ml, or is about 3 ml, or is about 5 ml. Veterinarily acceptable carriers suitable for use in immunogenic compositions and vaccines can be any of those described herein.

Those skilled in the art can readily determine whether a virus needs to be attenuated or inactivated before administration. In another embodiment, the recombinant PPV can be administered directly to an animal without additional attenuation. The amount of a virus that is therapeutically effective can vary depending on any of several factors including the condition of the animal and the degree of infection, and can be determined by a skilled artisan.

In accordance with the methods of the present invention, a single dose can be administered to animals, or, alternatively, two or more inoculations can take place with intervals of from about two to about ten weeks. Boosting regimens can be required and the dosage regimen can be adjusted to provide optimal immunization. Those skilled in the art can readily determine the optimal administration regimen.

Immunogenic compositions and vaccines can be administered directly into the bloodstream, into muscle, or into an internal organ. They can be administered orally or intranasally. Suitable means for parenteral administration include, but are not limited to, intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Parenteral formulations are typically aqueous solutions which can contain excipients such as salts, carbohydrates and buffering agents (preferably to a pH of from about 3 to about 9, or from about 4 to about 8, or from about 5 to about 7.5, or from about 6 to about 7.5, or about 7 to about 7.5), but, for some applications, they can be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, can readily be accomplished using standard pharmaceutical techniques well known to those skilled in the art.

The recombinant parapoxviruses and immunogenic compositions and vaccines described herein can be used in the preparation of a medicament for vaccinating an animal against canine distemper disease.

The present invention provides methods of determining the origin of a parapoxvirus present in an animal subject.

Vaccination which utilizes a DIVA vaccine—one which is able to differentiate infected from vaccinated animals—provides a means for determining the origin of a parapoxvirus present in an animal subject. This differentiation can be accomplished via any of various diagnostic methods, including but not limited to ELISA, Western blotting and PCR. These and other methods are readily recognized and known to one of ordinary skill in the art.

The parapoxviruses described herein can be distinguished from wild-type strains in both their genomic composition and proteins expressed. Such distinction allows for discrimination between vaccinated and infected animals. For example, a determination can be made as to whether an animal testing positive for parapoxvirus in certain laboratory tests carries a wild-type parapoxvirus strain, or carries a recombinant parapoxvirus previously obtained through vaccination.

A variety of assays can be employed for making the determination. For example, virus can be isolated from the animal testing positive for parapoxvirus, and nucleic acid-based assays can be used to determine the presence of a parapoxvirus genome, indicative of prior vaccination. The nucleic acid-based assays include Southern or Northern blot analysis, PCR, and sequencing. Alternatively, protein-based assays can be employed. In protein-based assays, cells or tissues suspected of an infection can be isolated from the animal testing positive for parapoxvirus. Cellular extracts can be made from such cells or tissues and can be subjected to, e.g., Western Blot, using appropriate antibodies against viral proteins that can distinctively identify the presence of either the recombinant parapoxvirus previously inoculated, or wild-type parapoxvirus.

The extent and nature of the immune responses induced in the animal can be assessed by using a variety of techniques. For example, sera can be collected from the inoculated animals and tested for the presence or absence of antibodies specific for the parapoxvirus e.g. in a conventional ELISA. Detection of responding cytotoxic T-lymphocytes (CTLs) in lymphoid tissues can be achieved by assays such as T cell proliferation, as indicative of the induction of a cellular immune response. The relevant techniques are well described in the art, e.g., Coligan et al. Current Protocols in Immunology, John Wiley & Sons Inc. (1994).

The recombinant Parapoxvirus ovis D1701-V-CDV-H can be used in a DIVA assay. In one embodiment, it can be used in assays for the detection of canine distemper N-genes or proteins to differentiate infected from vaccinated animals. In another embodiment, it can be used in assays for the detection of canine distemper P-genes or proteins to differentiate infected from vaccinated animals. In yet another embodiment, it can be used in assays for the detection of canine distemper L-genes or proteins to differentiate infected from vaccinated animals. In still another embodiment, it can be used in assays for the detection of canine distemper M-genes or proteins to differentiate infected from vaccinated animals.

The recombinant Parapoxvirus ovis Parapoxvirus ovis D1701-V-CDV-F can be used in a DIVA assay. In one embodiment, it can be used in assays for the detection of canine distemper N-genes or proteins to differentiate infected from vaccinated animals. In another embodiment, it can be used in assays for the detection of canine distemper P-genes or proteins to differentiate infected from vaccinated animals. In yet another embodiment, it can be used in assays for the detection of canine distemper L-genes or proteins to differentiate infected from vaccinated animals. In still another embodiment, it can be used in assays for the detection of canine distemper M-genes or proteins to differentiate infected from vaccinated animals.

The present invention is additionally described by the following illustrative, non-limiting Examples.

### EXAMPLES

### Example 1

### Generation of CDV H and CDV F Protein Expressing Recombinant Virus D1701-V-CDV-H and D1701-V-CDV-F

Recombinant Parapoxvirus ovis containing the gene encoding for the H protein of CDV, as well as recombinant Parapoxvirus ovis containing the gene encoding for the F protein of CDV, were generated. Expression of the H and F proteins were assessed.

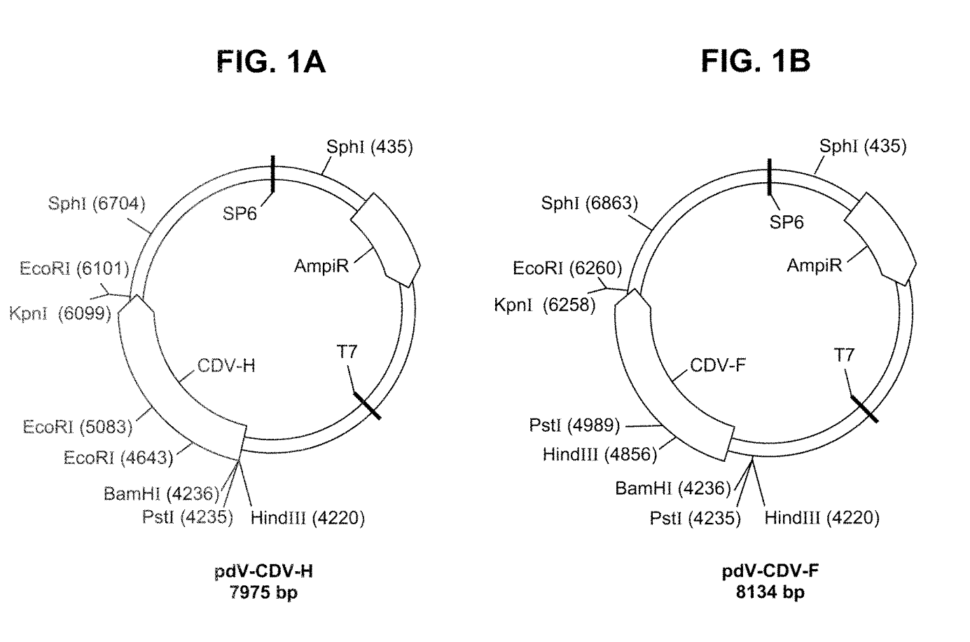

For generation of the recombinant Parapoxvirus ovis D1701-V-CDV-H and D1701-V-CDV-F, the Parapoxvirus ovis (PPVO) vector system (U.S. Pat. No. 6,365,393; Rziha at al., 2000, J. Biotechnol., 83, 137-145; Fischer et al., 2003, J. Viral. 77, 9312-9323; Henkel et al., 2005, J. Virol. 79, 314-325) was used. The CDV H gene and F gene were obtained by PCR from the virus strain Rockborn, and cloned as BamHI-KpnI DNA fragments of 1865 bp (H) or 2024 bp (F) in size, following BamHI-KpnI restriction digestion, agarose gel (0.8% w/v) electrophoresis, and purification by Qiaex II gel extraction (Qiagen; Germany). The plasmid pdV-Rec1 (Fischer et al., 2003) was double-digested with BamHI and KpnI and used for ligation (Fast ligation kit, Promega; Germany). After transformation of E. coli DH5αF (Invitrogen, Thermo Fisher Scientific; Germany) insert-positive colonies were selected by BamHI-KpnI restriction digestion of plasmid DNA. This resulted in transfer plasmid pdV-CDV-H (FIG. 1A), or in transfer plasmid pdV-CDV-F (FIG. 1B). Relevant restriction sites and their nucleotide positions in the plasmids are indicated in the Figure. Ampicillin resistance gene (AmpiR) and the T7 and SP6 promoters are also indicated. Both transfer plasmids were prepared by Qiagen Plasmid Maxi Kit (Qiagen; Germany) and used for DNA sequencing. To this end, primer ORF32N (SEQ ID NO: 5;

5′-GCGCGCTGCGGGTGCGCTACCAATTCGCGC-3′) located upstream of the insertion site and primer ORF31N (SEQ ID NO: 6;

5′-GCATCCCGTTACCACCGGAGACCGACGCTCCC-3′) located downstream of the insertion site in pdV-Rec1 was used as well as internal H-gene-specific primers CDH-F (SEQ ID NO: 7; 5′-CAGATGCAGTGGAGCTACTACTTC-3′) and CDH-R (SEQ ID NO: 8; 5′-GGATCTAGAGGTAATGTCAACCGC-3′), respectively. This allowed the determination of the complete sequence of the inserted H gene (SEQ ID NO: 1). The internal F-gene-specific primers CDF-F (SEQ ID NO: 9,5′-CCCTGCTATGCAACATATGTCGTGTG-3′) and CDF-R (SEQ ID NO: 10, 5′-CGTTATACCATATCAGGGTATTGCCTCC-3′) allowed determination of the complete F gene sequence (SEQ ID NO: 2).

### Selection of Recombinants by Bluo-Gal Staining,

Vero cells (10⁶ cells) were infected with 0.1 MOI (multiplicity of infection) of the lacZ-expressing virus D1701-VrV, and 2 hr later transfected with 2 μg of pdV-CDV-H or pdV-CDV-F plasmid DNA by nucleofection, according to the manufacturer's recommendation (Amaxa Nucleofactor: Lonza, Germany). Virus lysates were harvested 3-4 days later, and used for titration on Vero cells in 6-well plates (Fisher Scientific; Germany). When plaques became visible, agarose-containing Bluo-Gal was overlaid as described (Fischer et al., 2003). Virus plaques having a white appearance were picked, and the single plaque eluates (overnight at 4° C. in phosphate-buffered saline (PBS)) were used for simultaneous infection of Vero cells (1×10⁵ cells) in single wells of a 48-well plate.

### Selection of Recombinants by Plaque-PCR.

Isolation of DNA from each virus plaque isolate was performed in a modification of Pasamontes et al. (J. Virol. Methods 35:137-141; 1991). Virus lysate (0.2 ml) was frozen (−70° C.) and thawed (37° C.) three times, and sonicated on ice 3 times for 20-30 sec (sonic waterbath). After phenol and chloroform extraction, 10 μg yeast tRNA or 3 μl GlycoBlue (Ambion; Germany) were added before ethanol precipitation. The DNA pellet was washed twice with 70% (v/v) ethanol, and dissolved after drying in 14 μl aqua bidest.

For CDV H-specific PCR, 4 μl of the DNA were mixed on ice with 1 μl primer mix, consisting of 4.0 pmol CDH-F (SEQ ID NO: 7) and 4.0 pmol CDH-R (SEQ ID NO: 8) primers, and 5 μl ReddyMix 2×PCR (Abgene, Thermo Fisher Scientific; Germany). PCR was performed in a Trio Thermoblock (Biometra; Germany) by incubating for 2 min at 98° C., followed by 40 cycles for 1 min at 96° C., 30 sec at 65° C., 30 sec at 72° C., and a final extension step for 2 min at 72° C. Identical conditions were employed for CDV F-specific PCR, using primers CDF-F and CDF-R, respectively, except for 30 sec at 68° C. (instead of 65° C.). The PCR products were separated in horizontal 1% (w/v) agarose-ethidium bromide (0.3 microgram per ml) gels. Virus lysates from plaque isolates revealing the H gene-specific PCR fragment of 686 bp in size were diluted and further plaque-purified at least 3 times, using Bluo-Gal agarose overlay as described above. Plaque isolates from transfection with plasmid pdV-CDV-F, which showed the F gene-specific PCR fragment of 766 bp in size, were also further plaque-purified as described for the H gene-containing virus plaques. Finally, the DNA of recombinant virus plaque isolates positive for the H or for the F gene were tested in a LacZ gene-specific PCR using 4 μl DNA, 3.95 pmol primer lacZ-F (SEQ ID NO: 11; 5′-cgatactgtcgtcgtcccctcaa-3′), and 4.13 pmol primer lacZ-R (SEQ ID NO: 12; 5′-caactcgccgcacatctgaact-3′). After adding 5 μl AccuPrime SuperMix II (Invitrogen, Fisher Scientific; Germany), PCR was performed by heating for 2 min at 98° C., followed by 40 cycles for 1 min at 96° C., 30 sec at 62° C., and 90 sec at 68° C., with a final extension step for 2 min at 68° C. Separation of FOR products was performed as described above. The absence of the LacZ gene-specific fragment of 508 bp in size demonstrated that the corresponding recombinant virus plaque isolates were free of the LacZ-expressing parental virus D1701-VrV following three rounds of plaque purification. After preparation of high titer virus stocks of D1701-V-CDV-H and D1701-V-CDV-F, viral DNA was prepared as described below, and used for testing in CDV-PCR and LacZ-PCR.

### Immunohistochemical Staining of Recombinant Virus Plaques (IPMA).

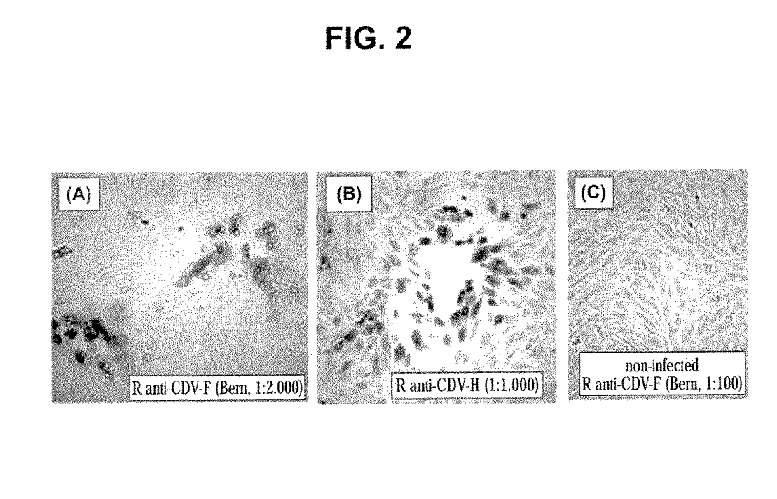

Successful expression of inserted foreign gene was first assayed by IPMA, which involves immunohistochemical staining of recombinant virus plaques titrated on Vero cells in 24-well plates. After the appearance of virus plaques, the medium was aspirated from each well, and the cells dried by leaving the plate open for approximately 10 min in a laminar flow hood. Thereafter, the cells were fixed with ice-cold absolute methanol at −20° C. for 15-20 min. After washing twice with ice-cold 1% (v/v) fetal calf serum (FCS) in PBS, the cells were blocked with PBS containing 10% (v/v) FCS for 90 min at room temperature (RT). Detection of foreign gene expression was achieved by incubation for 60 min at RT with a polyclonal rabbit (R) antiserum against the F protein (provided by P. Plattet & A. Zurbriggen; Univ. Bern, Switzerland), diluted 1:2.000 (FIG. 2A), or the polyclonal H protein-specific rabbit antiserum MC711 (provided by V. von Messling; Univ. Quebec, Canada), diluted 1:1.000 in 1% FCS in PBS (FIG. 2B). After 3 washes with PBS-T (PBS containing 0.05% (v/v) Tween-20), a peroxidase-coupled anti-rabbit secondary antibody (Jackson-ImmunoRes., DIANOVA; Germany), diluted 1:2000, was added, and incubated for 60 min at RT. After thorough washing with PBS-T and PBS, substrate (Vector Nova Red, Axxora; Germany) was added as recommended by the manufacturer's instruction, until red-brown positive staining became visible. As negative controls, non-infected cells were incubated with polyclonal rabbit antiserum, diluted 1:100, against the F protein (FIG. 2C). Cells infected with D-1701-VrV or D-1701-V were also used as negative controls. Virus plaques and infected cells were found strongly positive for the F protein (FIG. 2A) and the H protein (FIG. 2B) of CDV.

### Example 2

### Characterization of D1701-V-CDV-H and D1701-V-CDV-F

### Preparation of Virus Stocks.

To obtain high titer recombinant virus stocks of D1701-V-CDV-H and D1701-V-CDV-F, 10-20 T150 culture flasks (Greiner; Germany) were simultaneously infected with a MOI of 0.5. After 3 days, approximately 80% cytopathogenic effect (CPE) was observed, and the cells and supernatant of all flasks were harvested and collected for centrifugation (2 hr at 13,000 rpm, 4° C.). The supernatant was carefully removed, and the virus pellet was dissolved overnight at 4° C. in 1-2 ml PBS. The virus suspension was completely dispersed by sonification (Sonic cell disruptor, Branson; Germany) on ice using 3 pulses (100 W) of 10 sec, (10 sec break between each pulse), followed by centrifugation (500-700×g, 10 min, 4° C.) to remove cell debris. The supernatant was stored on ice, while the cell pellet was resuspended in 1.0 ml PBS, and sonicated on ice (2 times for 20 sec, with a 10 sec break in between, then once for 30 sec). After low speed centrifugation, the supernatant was combined with the first supernatant, divided into aliquots, titrated, and stored at −70° C.

### Characterization of Viral DNA.

Vero cells were infected with MOI 0.5, and harvested after 2-3 days (approx. 80% CPE) by trypsinisation and brief low speed centrifugation at 4° C. DNA was isolated using the Master Pure DNA Isolation Kit (Epicentre Biotechnology, Biozym Scientific; Germany), according to the manufacturer's protocol.

To verify insertion of the CDV H or the F gene in the correct locus, 2 μg DNA were restriction enzyme-digested, separated in 0.8% (w/v) agarose gels, and transferred to nylon membrane (GE Healthcare; Germany) for Southern blot hybridization according to standard procedures. A CDV H or F gene-specific probe (the product of the CDV-H or CDV-F PCR) was gel-isolated, radioactively labeled (³²P-dCTP, MP Biomedicals; Germany) using RediPrime (GE Healthcare; Germany). This was then used for Southern blot hybridization, carried out under conditions of 50° C. in 4×SSPE (1×=0.18 M NaCl, 10 mM PP, 1 mM EDTA, pH 7.4) containing 0.5% (w/v) non-fat milk powder, 1.0% (w/v) sodium dodecylsulfate (SDS), and 0.5 mg/ml denatured calf thymus DNA (KT-DNA, Sigma; Germany). Following X-ray (Kodak X-Omat; Germany) exposure, the probe was removed from the filter by incubation in 0.4 N NaOH at 45° C. for 30-60 min, followed by brief incubation at 100° C. in 0.1×SSC, 0.5% SDS, 0.2 M Tris-HCl (pH 7.4). For the second hybridization, the HindIII fragment H of D1701-V containing the vegF-E locus was used as described (Cottone et al., 1998. Virus Res. 56, 53-67). Southern blot results confirmed the correct insertion of the H or F gene into the genome of D1701-VrV.

### Detection of CDV H or F Gene-Specific RNA.

Vero cells were infected with a MOI of 3-5, and total RNA was isolated at different times after infection (p.i.) using SurePrep Total RNA Extraction Kit (Fisher Scientific; Germany). Additionally, RNA was extracted from cells infected in the presence of cytosine arabinoside (AraC; 0.04 Sigma; Germany) or cycloheximide (CHX, 0.1 mg/ml, Serva; Germany) to test for early expression of the inserted H- or F-gene. As a control, RNA was isolated from non-infected cells. The RNAs were separated in a denaturing 1% agarose gel containing formaldehyde, and transferred to nylon membrane as described (Kroczek, R. A. & Siebert, E. Anal. Biochem. 184:90-95, 1990). The radioactively-labeled CDV H or CDV F PCR fragment was used as hybridization probe in UltraHyb solution (Ambion; Germany) at 42° C. The results clearly demonstrated immediate early expression of the CDV-H or CDV-F gene, due to its regulation under the control of the early vegF-E promoter of ORFV (Rziha et al. 1999, J. Biotechnol., 73, 235-242).

### Detection of H or F Protein of CDV by Immunofluorescence.

For immunofluorescence, Vero cells (1×10⁵ cells/ml) were infected in 4-chamber slides (BD Falcon; Germany) with a MOI of 1.0. At different times p.i., the cells were washed with medium, and fixed with 3.7% (v/v) methanol-free formaldehyde (Pierce, Thermo Fisher Scientific; Germany) for 15 min at 37° C. After 3 washes with PBS, the cells were permeabilized by treatment with 0.2% (v/v) Triton X-100 for 5 min at 37° C. After PBS washing, the cells were blocked with 5% FCS in PBS for 30-40 min at 37° C. For CDV-H or CDV-F protein detection, cells were incubated for 1 hr at 37° C. with rabbit anti-CDV-H antibody (diluted 1:2000) or with rabbit anti-CDV-F antibody (diluted 1:200), and anti-rabbit-Alexa-555 (diluted 1:2000). After 5 washes in PBS; slides were incubated in the dark at 37° C. for 30 min with the secondary anti-rabbit Alexa-555 or anti-rabbit Alexa-488 antibody, diluted 1:2000 in PBS (Molecular Probes; Germany). As negative control, non-infected cells were used.

Detection of cells at late times after infection with ORFV was carried out by the use of ORFV-specific rabbit antiserum PAS2274, provided by Dr. Rudiger Raue, (Pfizer Inc, UK). The serum was diluted 1:100 in PBS with 1% FCS, and secondary antibody, the anti-rabbit Alexa-488, was used at a 1:2000 dilution.

Staining of the actin cytoskeleton was performed with Phalloidin-647, according to the instructions of the manufacturer (Biotium; Germany) followed by staining of the nucleus with 0.04 μg/ml DAPI (4′,6-Diamidin-2′-phenylindoldihydrochlorid: Roche Molecular Biochemicals; Germany), for 20-30 min at RT in the dark. After thorough washing in PBS, the slides were embedded with Mowiol-DABCO, and fluorescence imaging was performed with a Zeiss ApoTome, using Axiovision software. Nuclei were stained with DAPI.

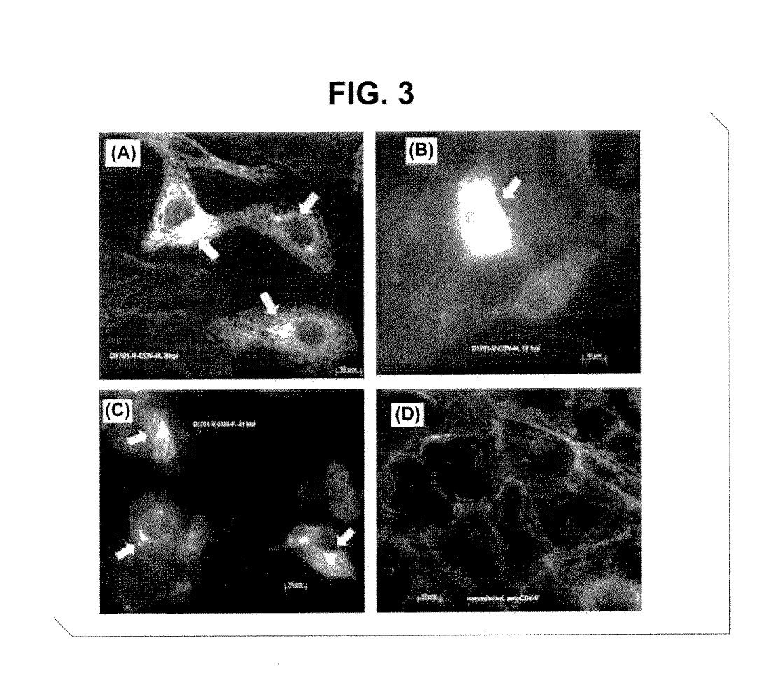

The results shown in FIG. 3 clearly demonstrate strong expression of the CDV H protein or CDV F protein in Vero cells infected with each recombinant (MOI=1.0). Cells infected late could be additionally identified by specific staining with antiserum PAS2274. Specificity of staining was tested via the use of non-infected cells or cells infected with D1701-V or D1701-VrV.

### Detection of CDV H or F Protein by Western Blotting.

Vero cells (3×10⁵ cells) were simultaneously infected with a MOI of 3.0, and incubated at 37° C. in a 5% CO₂ atmosphere. At different times p.i., the cells plus supernatant were harvested, centrifuged (8,900×g, 10 min, 4° C.), and the cell sediment was washed 3 times with 1.0 ml PBS and resuspended in 0.15 ml PBS containing 1% (v/v) Triton X-100. After 30 min on ice, the lysate was centrifuged for 15 min at 15.000×g, 4° C., and the supernatant saved for SDS-PAGE (Polyacrylamide gel electrophoresis). To this end, three parts of lysate were mixed with one part 4× DualColor protein loading buffer (Fermentas; Germany), boiled for 5 min, sonicated, and approx. 10 μg protein was separated by SDS-PAGE using 8% (w/v) ProSieve50 gel with Tris-Tricine-SDS running buffer as recommended (FMC Bioproducts, Biozym; Germany). The Prestained Protein Ladder (Fermentas; Germany) was used as molecular weight markers. After electrophoresis, the proteins were transferred to a PVDF membrane according to the instruction of the manufacturer (Pierce, Thermo Fisher Scientific; Germany). After membrane blocking in 3× Rotiblock (Roth; Germany) for 3 hours at room temperature, polyclonal rabbit anti-H or anti-F antiserum (provided by Dr. P. Plattet & Dr. A. Zurbriggen; Univ. Bern, Switzerland) was used 1:10,000, diluted in 1× RotiBlock. After overnight incubation at 4° C., the membrane was thoroughly washed 5 times in TBS-T (Tris-buffered saline with 0.05% v/v Tween-20), and incubated with a peroxidase-coupled anti-rabbit antibody (1:20,000; Jackson-ImmunoRes., Dianova; Germany) for 1 hr at RT. After TBS-T washing, ECL substrate was used as recommended (Immobilon Western, Millipore; Germany). The reacted proteins were detected by the use of chemiluminescence X-ray film (CL-XPosure, Pierce, Thermo Fisher Scientific; Germany).

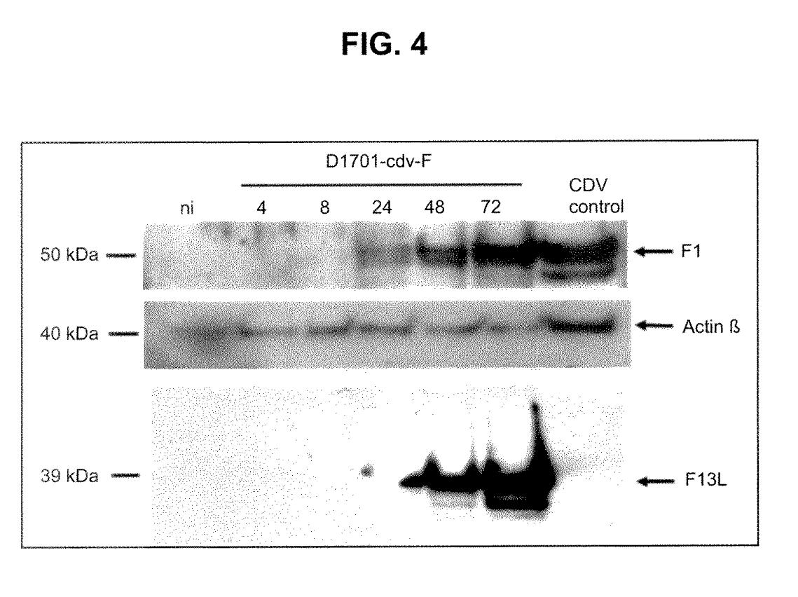

Expression of the CDV H or F protein of the expected molecular weight (H=80 kDa; F0=60 kDa, F1=40 kDa) was confirmed at the different times after infection. FIG. 4 representatively shows the detection of the F protein of CDV. The upper panel indicates reaction with the polyclonal F-specific rabbit antiserum, detecting the cleaved (F1) CDV gene product. The middle panel indicates detection of actin-β protein, demonstrating that comparable protein amounts have been loaded into each well. The lower panel indicates the detection of a late ORFV major envelope protein (F13L).

### In Vitro Virus Production.

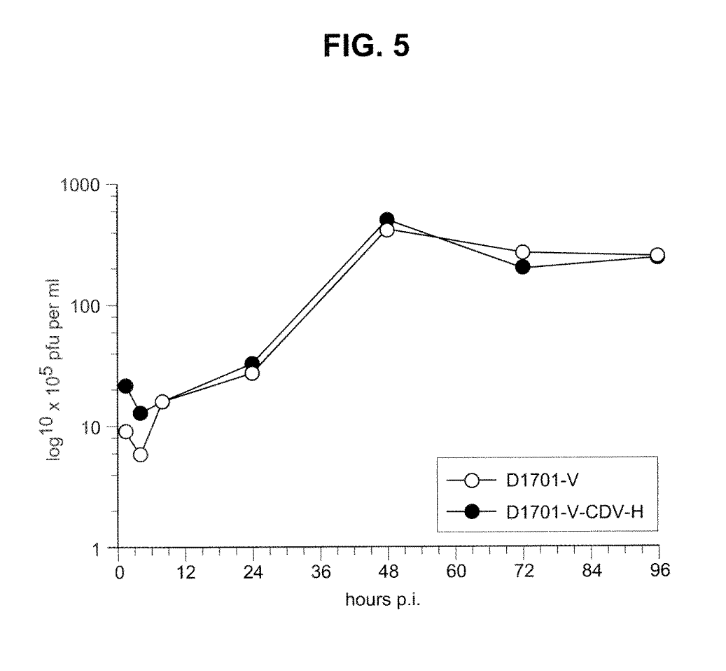

To test whether insertion of a CDV gene into the genome of the ORFV vector might impact virus growth, virus production of recombinants was compared with parental D1701-V by titration experiments, or Single-Step Virus Growth Curves (SSGC). As representatively shown for the D1701-V-CDV-H virus in FIG. 5, total cell lysates were harvested at the indicated hours after infection (p.i.), and titrated in triplicate. Production of infectious progeny was indistinguishable from the parental D1701-V virus. This experiment supports the conclusion that insertion of a CDV gene does not lead to altered in vitro growth characteristics compared to parental vector virus.

### Example 3

### Detection of CDV-H Specific Antibodies by Immunofluorescence

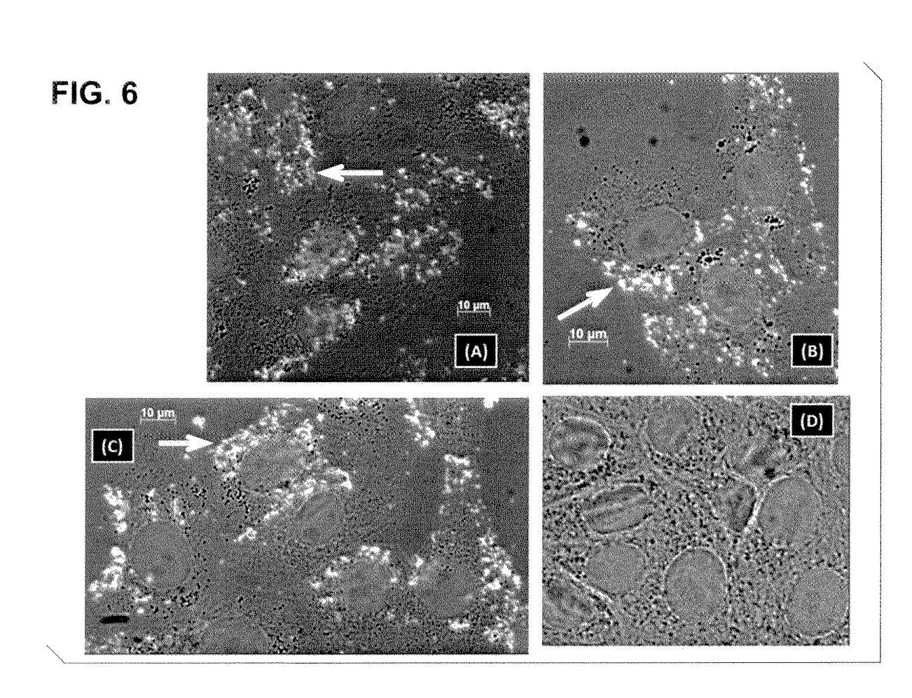

Vero cells were infected with the CDV strain Onderstepoort (FIGS. 6 A-C) or non-infected (FIG. 6 D), and fixed and incubated with serum (1:1000 diluted) of mice intramuscularly immunized with the recombinant D1701-V-CDV-H (10⁷ PFU).

Cell nuclei were stained by DAPI. The extranuclear staining is a result of specific reactivity with mouse antiserum. Panel D shows phase contrast illumination, and better visualizes CDV-infected cells characterized by cell fusions.

This assay demonstrated that immunization of mice with the ORFV recombinant expressing the CDV-H antigen led to the generation of specific serum antibodies.

### Example 4

### Detection of CDV-F Specific Antibodies by Immunofluorescence

Balb/C mice, 4-6 weeks of age (n=5 per group), are intramuscularly immunized either once, twice, or three times, in 2 weeks intervals, with 0.1 ml containing 10⁶ PFU or 10⁷ PFU of D1701-V-CDV-F. Individual serum samples are taken weekly until 2 weeks after the last immunization. To test the immunogenicity of D1701-V-CDV-F, the sera are analyzed by immunofluorescence and Western blotting according to the protocol described above.

Collectively, these results demonstrate the successful expression of the CDV H—in the ORFV recombinants D1701-V-CDV-H. They also demonstrate the successful expression of the F-gene in the ORFV recombinant D1701-V-CDV-F.

What is claimed is:

1. A recombinant parapoxvirus comprising a parapoxvirus and heterologous DNA derived from a canine distemper virus.

2. The recombinant parapoxvirus of claim 1, wherein the parapoxvirus is a Parapoxvirus ovis virus (ORFV).

3. The recombinant parapoxvirus of claim 2, wherein the parapoxvirus is Parapoxvirus ovis strain D1701.

4. The recombinant parapoxvirus of claim 3, wherein the recombinant parapoxvirus is selected from the group consisting of parapoxvirus ovis D1701-V-CDV-H and parapoxvirus ovis D1701-V-CDV-F.

5. The recombinant parapoxvirus of claim 1, wherein the heterologous DNA is selected from the group consisting of the gene encoding the H protein of the canine distemper virus, fragments of said gene encoding the H protein, the gene encoding the F protein of the canine distemper virus, and fragments of said gene encoding the F protein.

6. The recombinant parapoxvirus of claim 5, wherein the heterologous DNA is the gene encoding the H protein of the canine distemper virus, or fragments thereof.

7. The recombinant parapoxvirus of claim 5, wherein the heterologous DNA is the gene encoding the F protein of the canine distemper virus, or fragments thereof.

8. The recombinant parapoxvirus of claim 1, wherein the heterologous DNA is selected from the group consisting of SEQ ID NO: 1, a sequence having at least about 98% identity to SEQ ID NO: 1, SEQ ID NO: 2, and a sequence having at least about 98% identity to SEQ ID NO: 2.

9. The recombinant parapoxvirus of claim 1, wherein the heterologous DNA is inserted within the HindIII fragment H/H of Parapoxvirus ovis strain D1701.

10. The recombinant parapoxvirus of claim 9, wherein the heterologous DNA is inserted within the VEGF coding sequence or adjacent non-coding sequences within the HindIII fragment H/H of Parapoxvirus ovis strain D1701.

11. A method of preparing the recombinant parapoxvirus of claim 1 comprising inserting heterologous DNA into the genome of the parapoxvirus.

12. The method of claim 11, wherein the parapoxvirus is Parapoxvirus ovis.

13. The method of claim 11, wherein the parapoxvirus is Parapoxvirus ovis strain D 1701.

14. The method of claim 11, wherein the recombinant parapoxvirus is selected from the group consisting of parapoxvirus ovis D1701-V-CDV-H and parapoxvirus ovis D1701-V-CDV-F.

15. The method of claim 11, wherein the heterologous DNA is selected from the group consisting of the gene encoding the H protein of the canine distemper virus, fragments of said gene encoding the H protein, the F protein of the canine distemper virus, and fragments of said gene encoding the F protein.

16. The method of claim 15, wherein the heterologous DNA is the gene encoding the H protein of the canine distemper virus, or fragments thereof.

17. The method of claim 15, wherein the heterologous DNA is the gene encoding the F protein of the canine distemper virus, or fragments thereof.

18. The method of claim 11, wherein the heterologous DNA is selected from the group consisting of SEQ ID NO: 1, a sequence having at least about 98% identity to SEQ ID NO: 1, SEQ ID NO 2, and a sequence having at least about 98% identity to SEQ ID NO: 2.

19. An immunogenic composition comprising the recombinant parapoxvirus of claim 1 and a carrier.

20. The immunogenic composition of claim 19, wherein the parapoxvirus is Parapoxvirus ovis.

21. The immunogenic composition of claim 20, wherein the parapoxvirus is Parapoxvirus ovis strain D 1701.

22. The immunogenic composition of claim 19, wherein the recombinant parapoxvirus is selected from the group consisting of parapoxvirus ovis D1701-V-CDV-H and parapoxvirus ovis D1701-V-CDV-F.

23. The immunogenic composition of claim 19, wherein the heterologous DNA is selected from the group consisting of the gene encoding the H protein of the canine distemper virus, fragments of said gene encoding the H protein, the F protein of the canine distemper virus, and fragments of said gene encoding the F protein.

24. The immunogenic composition of claim 23, wherein the heterologous DNA is the gene encoding the H protein of the canine distemper virus, or fragments thereof.

25. The immunogenic composition of claim 23, wherein the heterologous DNA is the gene encoding the F protein of the canine distemper virus, or fragments thereof.

26. The immunogenic composition of claim 19, wherein the heterologous DNA is selected from the group consisting of SEQ ID NO: 1, a sequence having at least about 98% identity to SEQ ID NO: 1, SEQ ID NO: 2, and a sequence having at least about 98% identity to SEQ ID NO: 2.

27. A method of preparing the immunogenic composition of claim 19.

28. The method of claim 27, wherein the parapoxvirus is Parapoxvirus ovis.

29. The method of claim 28, wherein the parapoxvirus is Parapoxvirus ovis strain D 1701.

30. The method of claim 27, wherein the recombinant parapoxvirus is selected from the group consisting of parapoxvirus ovis D1701-V-CDV-H and parapoxvirus ovis D1701-V-CDV-F.

31. The method of claim 27, wherein the heterologous DNA is selected from the group consisting of the gene encoding the H protein of the canine distemper virus, fragments of said gene encoding the H protein, the F protein of the canine distemper virus, and fragments of said gene encoding the F protein.

32. The method of claim 27, wherein the heterologous DNA is selected from the group consisting of SEQ ID NO: 1, a sequence having at least about 98% identity to SEQ ID NO: 1, SEQ ID NO: 2, and a sequence having at least about 98% identity to SEQ ID NO: 2.

33. A method of inducing in an animal subject an immune response against canine distemper virus comprising administering to said animal a therapeutically effective amount of the immunogenic composition of claim 19.

34. The method of claim 33, wherein an anti-H or anti-F protein-specific protective immune response is induced.

35. A method of treating an animal subject against canine distemper disease, comprising administering to said animal a therapeutically effective amount of the immunogenic composition of claim 19.

36. Use of the recombinant parapoxvirus of claim 1 in the preparation of a medicament for treating an animal against canine distemper disease.

37. A use of the recombinant parapoxvirus of claim 1 in an assay for the differentiation of infected from vaccinated animals.

38. The use of claim 37, wherein the recombinant parapoxvirus is selected from the group consisting of parapoxvirus ovis D1701-V-CDV-H and parapoxvirus ovis D1701-V-CDV-F.
